(19) United States
(12) Patent Application Publication
(10) Pub. No.: US 2025/0367274 A1
(43) Pub. Date: Dec. 4, 2025

(54) Tolerizing Immune Modifying Nanoparticles for Overcoming the Immunogenicity of Therapeutic Vectors and Proteins

(71) Applicant: COUR PHARMACEUTICALS DEVELOPMENT COMPANY INC., Skokie, IL (US)

(72) Inventors: John Puisis, Northbrook, IL (US); Adam Elhofy, Chicago, IL (US); Joseph Podojil, Northbrook, IL (US); Michael Boyne, Skokie, IL (US); Tushar Murthy, Northbrook, IL (US)

(21) Appl. No.: 18/860,816

(22) PCT Filed: Apr. 28, 2023
(86) PCT No.: PCT/US23/20435

§ 371 Date: Oct. 28, 2024

Related U.S. Application Data
(60) Provisional application No. 63/336,754, filed on Apr. 29, 2022.
(60) Provisional application No. 63/343,797, filed on May 19, 2022.

(51) Int. Cl. A61K 39/00; A61K 9/00; A61K 9/51; C12N 15/86

(52) U.S. Cl. CPC A61K 39/001; A61K 9/0019; A61K 9/5153; C12N 15/86; A61K 2039/577; A61K 2039/6093; A61K 2039/64; C12N 2750/14143; C12N 2800/95

(57) ABSTRACT

The present application is directed, in general, to tolerizing immune mediated particles comprising gene therapy vector antigens for use in combination with gene therapy regimens in order to reduce immunogenicity to the gene therapy vector antigens and/or transgene protein products expressed by the vectors.

### CROSS-REFERENCE TO RELATED APPLICATIONS

The present application claims the priority benefit of U.S. Provisional Patent Application No. 63/336,754, filed Apr. 29, 2022 and U.S. Provisional Patent Application No. 63/343,797, filed May 19, 2022, herein incorporated by reference in their entireties.

### FIELD OF THE DISCLOSURE

The present application is directed, in general, to tolerizing immune mediated particles comprising gene therapy vector antigens for use in combination with gene therapy regimens in order to reduce immunogenicity to the gene therapy vector antigens and transgene protein products expressed by the vectors.

### BACKGROUND

Advancements in gene delivery vector design and development (e.g., retrovirus, lentivirus, adenovirus, and adeno associated virus (AAV) vectors) has led to gene therapies emerging as an attractive therapeutic option for a number of diseases and conditions, including rare and inherited genetic disorders, autoimmune disorders, neurodegenerative conditions, and cancer. Gene therapies rely on vector mediated delivery of exogenous DNA encoding therapeutic proteins such as enzymes, cytokines, and antibodies).¹⁻⁶ The goal of gene therapies is to enable sustained tissue-specific production of therapeutic proteins for long-term therapeutic efficacy.

In addition to production of proteins and enzymes for the treatment of rare and inherited genetic disorders (e.g., Hemophilia, Pompe Disease, Fabry Disease, and Mucopolysaccharidosis), gene therapy vectors are being used for tissue specific expression of autoantigens and tolerogenic cytokines/chemokines for the treatment of autoimmune diseases, cancer vaccines, and production of anti-tumor proteins, cytokines, chemokines, and antibodies for the treatment of cancers^{3,4,7}. Moreover, viral vectors, referred to as oncolytic viruses, have been engineered to lyse tumor cells and induce an anti-tumor immune response for the treatment of cancers^{8,9}.

Gene therapy vectors in use and currently under development for gene therapy applications are typically modified and/or engineered versions of naturally occurring retrovirus, lentivirus, adenovirus, and AAVs¹⁰ with several synthetic vectors also described in the literature¹¹. In order to achieve sustained therapeutic efficacy, viral vectors must be re-administered periodically^{19,20}.

The immunogenicity of gene therapy vectors as well as the therapeutic proteins expressed from the vectors presents a significant clinical challenge for periodic re-administration required for sustained therapeutic benefit as both the vector and the therapeutic protein being produced by the vector are cleared rapidly after administration. Additionally, a significant proportion of patients likely to benefit from gene therapies exhibit pre-existing immunity to gene therapy vectors making them ineligible for treatment¹²⁻¹⁴. Patients without pre-existing neutralizing antibodies are also impacted by the immunogenicity of vectors and therapeutic proteins as initial vector administration induces an immune response to the vector proteins which interferes with any re-administration required for sustained therapeutic benefit.

Current strategies aimed at preventing the immunogenicity of viral vectors rely on the concomitant administration of immunosuppressive regimens with significant side-effects such as increased risks of infections and even death¹⁵.

### SUMMARY OF THE DISCLOSURE

Induction of antigen-specific T cell tolerance to gene therapy vectors and transgene protein products produced by the vectors using TIMPs could potentially overcome immunogenicity issues associated with gene therapies enabling re-dosing and leading to improved therapeutic efficacy.

The present disclosure describes compositions and methods for inducing antigen-specific tolerance to gene therapy vectors and the transgene protein product produced by the vectors. Provided herein is a composition comprises a negatively charged carrier particle encapsulating an antigen, wherein the antigen is one or more gene therapy vector antigens and/or a portion thereof, or combinations of gene therapy vector antigens or portions thereof. In various embodiments, the antigen is a therapeutic protein produced by the gene therapy vector and/or a portion thereof, or one or more antigenic epitopes thereof. In various embodiments, the carrier particle comprises a polymer and has a negative zeta potential. In various embodiments, the polymer is a biodegradable polymer.

In various embodiments, the particle comprises a polymer selected from the group consisting of polyglycolic acid (PGA), polylactic acid (PLA), polysebacic acid (PSA), poly(lactic-co-glycolic) (PLGA), poly(lactic-co-sebacic) acid (PLSA), poly(glycolic-co-sebacic) acid (PGSA), polypropylene sulfide, poly(caprolactone), chitosan, a polysaccharide, or a lipid, polystyrene, diamond, a liposome, PEG, cyclodextran, a lipid or a metal such as Iron (Fe), zinc (Zn), cadmium (Cd), Gold, or Silver, or combinations thereof.

In various embodiments, the polymer is a co-polymer. In various embodiments, the co-polymer has varying molar ratios of constituent polymers. In various embodiments, the molar ratio is 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0.

In various embodiments, the carrier particle comprises poly (lactic-co-glycolic acid) (PLGA). In various embodiments, the particle comprises about 50:50, about 80:20 to about 100:0 polylactic acid: polyglycolic acid or from about 50:50, about 80:20 to about 100:0 polyglycolic acid: polylactic acid. In various embodiments, the particle comprises 50:50 polylactic acid: polyglycolic acid. In various embodiments, the particle comprises polylactic acid: polyglycolic acid from about 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0, including all values and ranges that lie in between these values.

In various embodiments, the particles have a negative zeta potential. In various embodiments, the zeta potential of the particles is from about −100 mV to about 0 mV, from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges therein. In various embodiments, the carrier particles have a negative zeta potential of between −30 mV to −80 mV. In various embodiments, the carrier particles have a negative zeta potential of between −30 mV to −60 mV. In various embodiments, the negative zeta potential is achieved by surface functionalization of the carrier particle. In various embodiments, the surface functionalization is carboxylation.

In various embodiments, the size, or diameter, of carrier particles is between 0.05 μm to about 10 μm. In various embodiments, the diameter of carrier particles is between 0.1 μm and about 10 μm. In various embodiments, the diameter of carrier particles is between 0.1 μm and about 5 μm. In various embodiments, the diameter of carrier particles is between 0.1 μm and about 3 μm. In various embodiments, the diameter of carrier particles is between 0.3 μm and about 5 μm. In various embodiments, the diameter of carrier particles is about 0.3 μm to about 3 μm. In various embodiments, the diameter of carrier particles is between about 0.3 μm to about 1 μm. In various embodiments, the diameter of carrier particles is between about 0.4 μm to about 1 μm. In various embodiments, the carrier particles have a diameter of about 100 to 10000 nm, about 100 to 5000 nm, about 100 to 3000 nm, about 100 to 2000 nm, about 100 to 1500 nm, about 300 to 5000 nm, about 300 to 3000 nm, about 300 to 1000 nm, about 300 to 800 nm, about 400 to 800 nm, or about 200 to 700 nm. In various embodiments, the carrier particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm, including all values and ranges therein. In various embodiments, the diameter of the negatively charged particle is between 400 nm to 800 nm. In various embodiments, the diameter of the negatively charged particle is between 350 nm to 800 nm.

In various embodiments, the carrier particles have a homogenous size distribution. In various embodiments, the carrier particles have a homogenous size distribution wherein at least 90% of the particles have a diameter of between 0.05 μm and about 10 μm, between 0.1 μm and about 10 μm, 0.1 μm and about 5 μm, 0.1 μm and about 3 μm, 0.3 μm and about 5 μm, 0.3 μm to about 3 μm including all values and ranges therein. In various embodiments, the carrier particles have a homogenous size distribution wherein at least 90% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm including all values and ranges therein. In various embodiments, the carrier particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein. In various embodiments, the carrier particles have a homogenous size distribution wherein at least 50% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm including all values and ranges therein. In various embodiments, the particles have a homogenous size distribution wherein at least 50% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm including all values and ranges therein. In various embodiments, the carrier particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein.

In various embodiments, the carrier particles have a homogenous size distribution wherein at least 10% of the particles have a diameter of between about 0.05 μm and about 10 μm, about 0.1 μm and about 10 μm, about 0.1 μm and about 5 μm, about 0.1 μm and about 3 μm, about 0.3 μm and about 5 μm, and about 0.3 μm and about 3 μm including all values and ranges therein. In various embodiments, the carrier particles have a homogenous size distribution wherein at least 10% of the particles have a diameter of about 100 to 10000 nm, about 100 to 5000 nm, about 100 to 3000 nm, about 100 to 2000 nm, about 300 to 5000 nm, about 300 to 3000 nm, about 300 to 1000 nm, about 300 to 800 nm, about 400 to 800 nm, or about 200 to 700 nm including all values and ranges therein. In various embodiments, the carrier particles have a diameter of about 50 nm, 100 nm, 200 nm, 300 nm, 400 nm, 500 nm, 600 nm, 700 nm, 800 nm, 900 nm, 1000 nm, 1100 nm, 1200 nm, 1300 nm, 1400 nm, 1500 nm, or 2000 nm including all values and ranges therein.

In various embodiments, the particle encapsulates an antigen, has a negative zeta potential of between −100 mV and 0 mV, and wherein the particle is between 100 and 1000 nm in diameter. In various embodiments, the particle encapsulates one or more antigens comprising one or more gene therapy vector antigens, portions thereof or combinations thereof, and wherein the size of the particle is between 400 and 800 nm and the particle has a negative zeta potential between −30 mV and −80 mV.

In various embodiments, the particle encapsulates an antigen, has a negative zeta potential of between −100 mV and 0 mV, and wherein the particle is between 100 and 1000 nm in diameter. In various embodiments, the particle encapsulates one or more antigens comprising one or more protein therapeutics produced by a gene therapy vector, portions or combinations thereof, and wherein the size of the particle is between 400 and 800 nm and the particle has a negative zeta potential between −30 mV and −80 mV.

In various embodiments, the particle encapsulates one or more gene therapy vector antigens, portions or combinations thereof, and one or more protein therapeutics produced by a gene therapy vector, portions or combinations thereof. In various embodiments, the antigen comprises one or more proteins, peptides or antigenic epitopes thereof.

Also contemplated herein is a composition comprising liposomes encapsulating one or more gene therapy vector antigens, portions, or combinations thereof.

In another embodiment, the disclosure provides a composition comprising liposomes encapsulating therapeutic proteins produced by the gene therapy vector and/or a portion thereof.

In various embodiments, the liposome is negatively charged. In various embodiments, the liposome has a negative zeta potential. In various embodiments, the negative zeta potential is between −100 mV to 0 mV. In various embodiments, the negatively charged liposomes have a zeta potential from about −100 mV to about 0 mV, from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges therein. In various embodiments, the liposomes have a negative zeta potential of between −30 mV to −80 mV. In various embodiments, the liposomes have a negative zeta potential of between −30 mV to −60 mV.

In various embodiments, the liposome is between 100-1000 nm, or between 300-800 nm or between 400-800 nm. In various embodiments, the liposome is about 100 nm, about 200 nm, about 300 nm, about 400 nm, about 500 nm, about 600 nm, about 700 nm, about 800 nm, about 900 nm or about 1000 nm.

In various embodiments, the antigen comprises a gene therapy vector antigen or one or more portions thereof. In various embodiments, the gene therapy vector is a viral vector. In various embodiments, the viral vector is selected from the group consisting of adenovirus, adeno-associated virus (AAV), herpes simplex virus, hepatitis B virus, lentivirus, retrovirus, alphavirus, flavivirus, rhabdovirus, measles virus, Newcastle disease virus, Coxsackievirus, transfusion transmitted viruses, anellovirus, human papilloma virus, poxvirus, vaccinia virus, modified Ankara virus, vesicular stomatitis virus, picornavirus, tobacco mosaic virus, cowpea mosaic virus, cowpea chlorotic mottle virus, physalis mosaic virus, Red clover necrotic mosaic virus, potato virus x, comovirus, chicken anemia virus or cucumber mosaic virus. In various embodiments, the virus is an oncolytic virus. In various embodiments the virus is a chimeric virus, a synthetic virus, a mosaic virus, or a pseudotyped virus.

In various embodiments, the particle encapsulates an antigen, wherein the antigen is one or more AAV vectors, portions or combinations thereof. In various embodiments, the AAV is selected from the group consisting of AAV-1, AAV-2, AAV-3, AAV-4, AAV-5, AAV-6, AAV-7, AAV-8, AAV-9, AAV-10, AAV-12, Anc80, a synthetic AAV, combinations or engineered versions thereof. In various embodiments, the antigen is one or more AAV capsid proteins. In various embodiments, the AAV capsid protein is VP-1, VP-2, and VP-3.

In various embodiments the gene therapy vector is a bacteria, bacteriophage, yeast, exosome, or erythrocyte.

In various embodiments, the gene therapy vector is used in the treatment of cancer, autoimmune disease, allergy, cardiovascular disease, metabolic disease, diabetes, enzyme deficiency, protein deficiency, cystic fibrosis, hematologic disorder, beta-thalassemia, sickle-cell disease, Hemophilia A, Hemophilia B, lysosomal storage disease, Fabry disease, Gaucher disease, Pompe disease, Niemann-Pick disease, Tay-Sachs disease, macular degeneration, mucopolysaccharidosis, venous thrombosis, von Willebrand disease, purpura fulminans, growth-hormone deficiency, gangliosidosis, alkaline hypophosphatasia, cholesterol ester storage disease, hyperuricemia, Duchenne Muscular Dystrophy, Huntington's disease, Parkinson's, Alzheimer's disease, choroiderimia, Stargardt Disease, Batten disease, spinocerebellar ataxia, ALS, frontotemporal lobar degeneration, ornithine transcarbamylase deficiency, retinitis pigmentosa, RPE-65 mutation-associated diseases, epidermolysis bullosa, recessive dystrophic epidermolysis bullosa, spinal muscular atrophy, phenylketonuria (PKU), X-linked myotubular myopathy, Crigler-Najjar syndrome, catecholaminergic polymorphic ventricular tachycardia, glycogen storage disease type 1, alpha-mannosidosis, Fragile X-syndrome, arginase deficiency, X-linked chronic granulomatous disease, adenosine deaminase deficiency, Leber's congenital amaurosis, lipoprotein lipase deficiency, cerebral adrenoleukodystrophy, metachromatic leukodystrophy, Fanconi anemia, achromatopsia, scleroderma, Osteogenesis imperfecta, coronary artery disease, tyrosinemia, peripheral neuropathy, optic neuropathy, coronary artery disease, respiratory syncytial virus (RSV)-mediated lower respiratory tract disease, Danon disease, severe leukocyte adhesion deficiency, pyruvate kinase deficiency, Charcot Marie Tooth disease, Wiskott Aldrich syndrome, alpha-synuclein tauopathies, refractory angina due to myocardial ischemia, myotonic dystrophy type I, claudication, peripheral artery disease, methylmalonic acidemia, sucrase-isomaltase deficiency, Niemann-Pick type B disease, α1-PI deficiency, hereditary angioedema, fibrinogen deficiency, Factor VIIa deficiency, Factor X deficiency, Factor XI deficiency, Factor XII deficiency, Protein C deficiency, anti-thrombin III deficiency, MPS I, MPS II, MPS III, MPS IV, MPS VI, MPS VII, MPS IX, calcification/ossification disorders, ENPP1 deficiency, ENPP3 deficiency, ABCC6 deficiency, aromatic L-amino acid decarboxylase deficiency, Angelman syndrome, hyperphenylalaninaemia, dementia, and Rett syndrome and Usher syndrome.

In various embodiments, the autoimmune disease is selected from the group consisting of multiple sclerosis, Addison's disease, ankylosing spondylitis, alopecia, osteoarthritis, psoriatic arthritis, scleroderma, type-I diabetes, rheumatoid arthritis, thyroiditis, systemic lupus erythematosus, Reynaud's syndrome, Behcets syndrome, Sjorgen's syndrome, autoimmune uveitis, Eaton Lamberts disease, autoimmune myocarditis, inflammatory bowel disease, Amyotrophic Lateral Sclerosis (ALS), Systemic Lupus, Neuromyelitis Optica, Idiopathic Thrombocytopenia Purpura, Thrombotic Thrombocytopenia Purpura, Membranous Nephropathy, Bullous Phemphigoid, Phemphigus Vulgaris, Myasthenia Gravis, Celiac disease, ulcerative colitis, Crohn's disease, erythema nodosa, glomerulonephritis, Goodpasture's syndrome, granulomatosis, Grave's disease, Guillain-Barre syndrome, Hashimoto disease, hemolytic anemia, Kawasaki Disease, mixed connective tissue disease, multifocal motor neuropathy, peripheral biliary cirrhosis, polyangiitis overlap syndrome, scleroderma type 1, sclerosis cholangitis, Stiffman syndrome, Takayasu arteritis, vitiligo or Wegeners granulomatosis.

In various embodiments, the allergy is a food allergy. In various embodiments, the allergy is an environmental allergy. In various embodiments, the food allergy is a peanut allergy, tree nut allergy, milk allergy, egg allergy, fish allergy, wheat allergy, celery allergy, or peach allergy. In various embodiments, the environmental allergy is pollen allergy, dust allergy, pet dander allergy, or mold allergy. In various embodiments, the pet allergy is cat allergy or dog allergy.

In various embodiments, the gene therapy vector is used for the treatment of cancer. In various embodiments the cancer selected from the group consisting of brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, testicular cancer, leukemia, lymphoma, melanoma and mesothelioma.

In various embodiments, the therapeutic produced by the gene therapy vector is a protein, a polypeptide, or a peptide. In various embodiments, the protein is a cytokine, a chemokine, a hormone, a growth factor, an enzyme, or an antibody.

In various embodiments, the antibody is a monoclonal antibody. In various embodiments, the antibody is a recombinant monoclonal antibody. In various embodiments, the antibody is selected from the group consisting of an antibody-protein fusion, an immunoadhesin, a monospecific antibody, a bispecific antibody, a trispecific antibody. In various embodiments, the antibody is selected from the group consisting of an antibody fragment, an antigen binding fragment (Fab), single-chain variable fragment (scFv). In various embodiments the antibody is a single-domain antibody. In various embodiments, the antibody is IgA, IgD, IgE, IgM, and/or variants thereof. In various embodiments, the antibody is targeted to IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TNF-α, TNF-β, TGF-β1, TGF-β2, TGF-β3, LT-β, 4-1BBL, APRIL, GITRL, LIGHT, OX40L, TALL-1, TRAIL, TWEAK, TRANCE, CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163,CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10R, IL-12Rβ1, IL-12Rβ2, IL-13Rα1, IL-13Rα2, IL-15Rα, IL-21R, IL-23R, IL-27Rα, IL-31 Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α,β,γ,δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof.

In various embodiments, the antibody targets one or more immune cells. In various embodiments, the immune cells are T-cells, B-cells, NK cells, NK-T cells, monocytes, macrophages, neutrophils, granulocytes, dendritic cells, mast cells, eosinophils, basophils, TH1 cells, TH2a cells, Treg cells, Tr1 cells, and Breg cells.

In various embodiments, the antibody is selected from the group consisting of abciximab, adalimumab, alemtuzumab, avelumab, azetolizumab, basiliximab, bevacizumab, bezlotoxumab, blinatumomab, canakinumab, certolizumab, cetuximab, daclizumab, denosumab, durvalumab, efalizumab, emicizumab, etokimab, golimumab, ipilimumab, ixekizumab, infliximab, natalizumab, nivolumab, olaratumab, omalizumab, ofatimumab, palivizumab, panitumumab, pembrolizumab, ramucirumab, rituximab, tocilizumab, trastuzumab, tremelimumab, secukinumab, ustekinumab, and vedolizumab

In various embodiments the therapeutic produced by the vector is a cytokine or a chemokine. In various embodiments the cytokines and chemokines are selected from the group consisting of IL-1α, IL-1β, IL-2, IL-3, IL-4, IL-5, IL-6, IL-7, IL-8, IL-9, IL-10, IL-11, IL-12, IL-12p70, IL-13, IL-14, IL-15, IL-16, IL-17, IL-17, IL-18, IL-20, IL-21, IL-22, IL-23, IL-24, IL-25, IL-26, IL-27, IL-27b, IL-28, IL-29, IL-30, IL-31, IL-32, IL-33, IL-35, IL-36, CCL1, CCL2, CCL3, CCL4, CCL5, CCL6, CCL7, CCL8, CCL9, CCL10, CCL11, CCL12, CCL14, CCL15, CCL16, CCL17, CCL18, CCL19, CCL20, CCL21, CCL22, CCL23, CCL24, CCL25, CCL26, CCL27, CCL28, CXCL1, CXCL2 (MCP-1), CXCL3 (MIP-1α, CXCL4 (MIP-1β, CXCL5 (RANTES), CXCL6, CXCL7, CXCL8, CXCL9, CXCL10, CXCL11, CXCL12, CXCL13, CXCL14, CXCL15, CXCL16, CXCL17, GM-CSF, IFN-α, IFN-β, IFN-γ, TNF-α, TNF-α, TNF-β, TGF-β1, TGF-β2, TGF-β3, LT-β, 4-1BBL, APRIL, GITRL, LIGHT, OX40L, TALL-1, TRAIL, TWEAK, and TRANCE.

In various embodiments, the protein is a hormone selected from the group consisting of adrenaline, melatonin, noradrenaline, norepinephrine, triiidithryonine, thyroxine, dopamine, prostaglandins, leukotrienes, prostacyclin, thromboxane, amylin, Anti-Müllerian hormone, adiponectin, adrenocorticotropic hormone, angiotensinogen, angiotensin, antidiuretic hormone, vasopressin, arginine vasopressin, calcitonin, cholecystokinin, corticotropin-releasing hormone, cortistatin, enkephalin, endothelin, erythropoieitin, follicle-stimulating hormone, galanin, gastric inhibitory peptide, gastrin, ghrelin, glucagon, glucagon-like peptide 1, gonadotropin releasing hormone, growth releasing hormone, hepcidin, Human chorionic gonadotropin, human placental lactogen, growth hormone, inhibin, insulin, insulin-like growth factor, leptin, lipotropin, leuteinizing hormone, melanocyte stimulating hormone, motilin, orexin, osteocalcin, oxytocin, peancreating polypeptide, parathyroid hormone, pituitary adenylate cyclase-activating peptide, prolactin, prolactin-releasing hormone, relaxin, renin, secretin, somatostatin, thrombopoietin, thyroid-stimulating hormone, thyrotropin-releasing hormone, vasoactive intestinal peptide, guanylin, uroguanylin, testosterone, dehydroepiandrosterone, androstenedione, dihydrotestosterone, aldosterone, estradiol, estrone, estriol, cortisol, progesterone, calcitriol, and calcisiol.

In various embodiments, the protein is a growth factor selected from the group consisting of adrenomedullin, angiopoietin, autocrine motility factor, bone morphogenetic proteins, ciliary neurotrophic factor, leukemia inhibitory factor, colony-stimulating factor, macrophage colony-stimulating factor, granulocyte colony-stimulating factor, granulocyte macrophage colony-stimulating factor, epidermal growth factor, ephrin A1, ephrin A2, ephrin A3, ephrin A4, ephrin A5, ephrin B1, ephrin B2, ephrin B3, erythropoietin, fibroblast growth factor, fibroblast growth factor 1, fibroblast growth factor 2, fibroblast growth factor 3, fibroblast growth factor 4, fibroblast growth factor 5, fibroblast growth factor 6, fibroblast growth factor 7, fibroblast growth factor 8, fibroblast growth factor 9, fibroblast growth factor 10, fibroblast growth factor 11, fibroblast growth factor 12, fibroblast growth factor 13, fibroblast growth factor 14, fibroblast growth factor 15, fibroblast growth factor 16, fibroblast growth factor 17, fibroblast growth factor 18, fibroblast growth factor 19, fibroblast growth factor 20, fibroblast growth factor 21, fibroblast growth factor 22, fibroblast growth factor 23, fetal bovine somatotrophin, glial cell line-derived neurotrophic factor, neurturin, persephin, artemin, growth differentiation factor −9, hepatocyte growth factor, hepatoma-derived growth factor, insulin-like growth factor −1, insulin-like growth factor −2, keratinocyte growth factor, migration-stimulating factor, macrophage-stimulating protein, myostatin, neuregulin 1, neuregulin 2, neuregulin 3, neuregulin 4, brain-derived neurotrophic factor, nerve growth factor, neurotrophin-3, neurotrophin-4, placental growth factor, platelet-derived growth factor, renalase, T-cell growth factor, thrombopoietin, transforming growth factors, transforming growth factor alpha, transforming growth factor beta, tumor necrosis factor-alpha, or vascular endothelial growth factor. In various embodiments the cytokine selected from the group consisting miscellaneous hematopoietins, Epo, Tpo, Flt-3L, SCF, M-CSF, and MSP.

In various embodiments, the protein is a thrombolytic agent selected from the group consisting of tissue plasminogen activator, streptokinase, urokinase, anistreplase, reteplase, kabikikinase, tenecteplase, and rokinase.

In various embodiments the therapeutic produced by the vector is an enzyme. In various embodiments the enzyme is selected from the group consisting of gene-editing nuclease enzymes, meganucleases, homing endonucleases, zinc-finger nucleases, transcription activator-like effector nucleases (TALENs), CRISPR-associated nucleases, and CRISPR-Cas9.

In various embodiments, the Cas protein is selected from the group consisting of Cas 3, Cas8a, Cas5, Cas8b, Cas8c, Cas 9, Cas10d, Cse1, Cse2, Csy1, Csy2, Csy3, GSU0054, Cas10, Csm2, Cmr5, Cas10, Csx11, Csx10, Csf1, Cas9, Csn2, Cas4, Cas12, Cas12a, Cas12b, Cas12c, Cas12d, Cas12e, Cas12f, Cas14, C2c10, Cas12g, Cas12h, Cas12i, Cas12k, C2cr4, C2cr8, C2cr9, Cas13, Cas13a, Cas13b, Cas13c, Cas13d. In various embodiments, the TALEN is a FokI-based, PvuII-based, I-TevI-based, IAniI-based, I-OnuI-based, or a MutH-based TALEN. In various embodiments, the enzyme is a nuclease selected from the group consisting of LAGLIDADG (meganuclease), GIY-YIG, His-Cys box, H-N-H, PD-(D/E)-xK, Vrs-like, megaTAL.

In various embodiments, the enzyme is selected from the group consisting of imiglucerase, taliglucerase alfa, velaglucerase alfa, β-glucocerebrosidase, alglucerase, agalsidase beta, agalsidase alpha, sebelipase alpha, alpha-L-iduronidase, human iduronate-2-sulfatase, N-sulphoglucosamine sulphohydrolase, elosulfase alpha, galsulfase, alpha-glucosidase (GAA), human alpha mannosidase, Factor VIII, Factor IX, beta-galactosidase, arginase, dystrophia myotonica-protein kinase, ornithine transcarbamylase, NADPH oxidase, NADH dehydrogenase 4, adenosine deaminase, lipoprotein lipase, beta-glucocerebrosidase, myotubularin, arylsulfatase A, DOPA decarboxylase, matrix metallopeptidase 1, fumaryl acetoacetate hydrolase, phenylalanine hydroxylase, pyruvate kinase, porphobilinogen deaminase, alkaline phosphatase, sucrase-isomaltase, acid sphingomyelinease, heparan sulfase sulfatase, N-acetylgalactosamine-6-sulfatase, N-acetylgalactosamine-4-sulfatase, alpha-1 proteinase inhibitor, 1-esterase inhibitor, fibrinogen, Factor VIIa, Factor X, Factor XI, Factor XII, Protein C, anti-thrombin III, ectonucleotide pyrophosphatase/phosphodiesterase 1, ectonucleotide pyrophosphatase/phosphodiesterase 3, and aromatic L-amino acid decarboxylase.

In various embodiments, the enzyme is a protease. In various embodiments, the protease is an aspartic protease, a cysteine protease, a metalloprotease, a serine protease, and/or a threonine protease. In various embodiments, the protease is selected from the group consisting of ADAM1, ADAM2, ADAM7, ADAM8, ADAM9, ADAM10, ADAM11, ADAM12, ADAM15, ADAM17, ADAM18, ADAM19, ADAAM20, ADAM21, ADAM22, ADAM23, ADAM28, ADAM29, ADAM30, ADAM33, heparinase, MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28.

In various embodiments, the therapeutic produced by the vector is a protein. In various embodiments, the protein is selected from the group consisting of photoactivatable channel rhodopsin protein, tumor protein 53, HIV Gag proteins, fragile X mental retardation protein, beta globin, Fibroblast Growth Factor 4, human growth factor, Wisckott Aldrich syndrome protein, survival motor neuron protein 1, survival motor neuron protein 2, retinal pigment epithelium (RPE) 65, vascular endothelial growth factor (VEGF), Rab escort protein (REP) 1, adrenoleukodystrophy protein, Fanconi anemia complementation group A protein, ATP-binding cassette transporters, Collagen VII (COL7) protein, Type I collagen, Type III and V collagen, β2 integrins, Lysosome-associated membrane protein, dystrophin, mini-dystrophin, insulin-like growth factor, T Cell Immune Regulator 1/ATPase H+ Transporting VO Subunit A3Pancreatic And Duodenal Homeobox 1, MAF BZIP Transcription Factor A, cystic fibrosis transmembrane conductance regulator protein, ATP binding cassette subfamily C member 6, ATP binding cassette subfamily A member 4, vascular endothelial growth factor A, serpin family A member 1, progranulin, microtubule-associated protein tau, C9orf72 protein and gap junction β2.

The present disclosure provides methods of inducing antigen specific immune tolerance using TIMPs encapsulating antigens comprising administering to a subject a composition comprising negatively charged particles encapsulating an antigen, wherein the antigen is one or more gene therapy vector antigens and/or one or more transgene protein products produced by the gene therapy vector(s), portions thereof, or combinations thereof. In various embodiments, the antigen is one or more gene therapy vector antigens, portions thereof, or combinations thereof, and/or transgene protein produced by the vector, portions, or combinations thereof. In various embodiments, administration of TIMPs encapsulating one or more antigens to a subject in need thereof induces antigen specific tolerance.

Also provided is a method of inducing tolerance in a subject in need thereof comprising administering to the subject a composition comprising a liposome as described herein.

In various embodiments, subjects are administered TIMPs encapsulating one or more gene therapy vector antigens in combination with TIMPs encapsulating one or more transgene protein products produced by the gene therapy vector(s). Administering TIMPs encapsulating gene therapy vector antigens in combination with TIMPs encapsulating transgene protein products produced by the gene therapy vector(s) induces antigen specific tolerance to both the gene therapy vector antigen and transgene protein products produced by the gene therapy vector. In various embodiments, subjects are administered TIMPs encapsulating one or more gene therapy vector antigens before, concomitantly, or after subjects are administered TIMPs encapsulating one or more transgene protein products produced by the gene therapy vector(s).

In various embodiments, the TIMPs encapsulating one or more gene therapy vector antigens are administered 5, 10, 15, 30, 45, or 60 minutes prior to the administration of TIMPs encapsulating one or more transgene protein products produced by the gene therapy vector(s). In various embodiments, the TIMPs encapsulating one or more gene therapy vector antigens are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of TIMPs encapsulating one or more transgene protein products produced by the gene therapy vector(s). In various embodiments, the TIMPs encapsulating one or more gene therapy vector antigens are administered 1, 2, 3, 4, 5, 6, or 7 days prior to the administration of TIMPs encapsulating one or more transgene protein products produced by the gene therapy vector(s). In various embodiments, the TIMPs encapsulating one or more gene therapy vector antigens are administered one week, two weeks, three weeks, four weeks, 1 month, 2 months, 3 months, 4 months, 5 months, 6 months, 7 months, 8 months, 9 months, 10 months, 11 months, or 12 months prior to the administration of TIMPs encapsulating one or more transgene protein products produced by the gene therapy vector(s).

In various embodiments, the TIMPs encapsulating one or more gene therapy vector antigens are administered 5, 10, 15, 30, 45, or 60 minutes after the administration of TIMPs encapsulating one or more transgene protein products produced by the gene therapy vector(s). In various embodiments, the TIMPs encapsulating one or more gene therapy vector antigens are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of TIMPs one or more encapsulating transgene protein products produced by the gene therapy vector(s). In various embodiments, the TIMPs encapsulating one or more gene therapy vector antigens are administered 1, 2, 3, 4, 5, 6, or 7 days after the administration of TIMPs encapsulating one or more transgene protein products produced by the gene therapy vector(s). In various embodiments, the TIMPs encapsulating one or more gene therapy vector antigens are administered one week, two weeks, three weeks, four weeks, 1 month, 2 months, 3 months, 4 months, 5 months, 6 months, 7 months, 8 months, 9 months, 10 months, 11 months, or 12 months after the administration of TIMPs encapsulating one or more transgene protein products produced by the gene therapy vector(s).

In various embodiments, the TIMPs are administered intravenously, subcutaneously, intramuscularly, intraperitoneally, intranasally, or orally. In various embodiments, the TIMPs are administered prior to, concomitantly, or after the administration of the gene therapy vector.

In various embodiments, the TIMPs are administered 5, 10, 15, 30, 45, or 60 minutes prior to the administration of the gene therapy vector. In various embodiments, the TIMPs are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of the gene therapy vector. In various embodiments, the TIMPs are administered 1, 2, 3, 4, 5, 6, or 7 days prior to the administration of the gene therapy vector. In various embodiments, the TIMPs are administered one week, two weeks, three weeks, four weeks, 1 month, 2 months, 3 months, 4 months, 5 months, 6 months, 7 months, 8 months, 9 months, 10 months, 11 months, or 12 months prior to the administration of the gene therapy vector.

In various embodiments, the TIMPs are administered 5, 10, 15, 30, 45, or 60 minutes after the administration of the gene therapy vector. In various embodiments, the TIMPs are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of the gene therapy vector. In various embodiments, the TIMPs are administered 1, 2, 3, 4, 5, 6, or 7 days after the administration of the gene therapy vector. In various embodiments, the TIMPs are administered one week, two weeks, three weeks, four weeks, 1 month, 2 months, 3 months, 4 months, 5 months, 6 months, 7 months, 8 months, 9 months, 10 months, 11 months, or 12 months after to the administration of the gene therapy vector.

In various embodiments, administering the TIMPs in a subject in need thereof induces antigen specific tolerance to the gene therapy vector antigen, portions thereof, or combinations thereof. In various embodiments, administering the TIMPs in a subject in need thereof induces antigen specific tolerance to the transgene protein product, portions, or combinations thereof. In various embodiments, administering the TIMPs in a subject reduces the inflammatory immune response to the gene therapy vector and/or the transgene protein product expressed by the vector. In various embodiments, administering the TIMPs in a subject reduces the inflammatory immune response to one or more encapsulated antigens. In various embodiments, administering the TIMPs in a subject in need thereof reduces the inflammatory response to the one or more antigens not encapsulated within the particle. Such an immune regulatory response has been referred to in the literature as ‘infectious tolerance’. In various embodiments, the inflammatory immune response is a humoral immune response and/or an adaptive immune response. In various embodiments, the immune response is an antibody response. In various embodiments, the antibody response is an IgA, IgG, IgE, or IgM response. In various embodiments, the antibody response is a neutralizing antibody response, for example the formation of neutralizing antibodies against the gene therapy vector antigen and/or the transgene protein produced by the vector. In various embodiments, the immune response is a T cell, B cell, NK cell monocyte, macrophage, eosinophil, or a basophil response. In various embodiments, the immune response is reduced immune infiltrate into tissues and/or organs.

In various embodiments, administering the TIMPs in a subject in need thereof induces an antigen specific immune regulator response. In various embodiments, administering the TIMPs in a subject induces regulatory T cells, B cells, monocytes, and/or macrophages. In various embodiments, administering TIMPs in a subject induces antigen specific regulatory T cell (Treg), Tr1, regulatory macrophages (Mreg), and/or regulatory B cells (Breg) cells.

In various embodiments, subjects are administered liposomes encapsulating one or more gene therapy vector antigens in combination with liposomes encapsulating one or more transgene protein products produced by the gene therapy vector(s). Administering liposomes encapsulating gene therapy vector antigens in combination with liposomes encapsulating transgene protein products produced by the gene therapy vector(s) induces antigen specific tolerance to both the gene therapy vector antigen and transgene protein products produced by the gene therapy vector. In various embodiments, subjects are administered liposomes encapsulating one or more gene therapy vector antigens before, concomitantly, or after subjects are administered liposomes encapsulating one or more transgene protein products produced by the gene therapy vector(s).

In various embodiments, the liposomes encapsulating one or more gene therapy vector antigens are administered 5, 10, 15, 30, 45, or 60 minutes prior to the administration of liposomes encapsulating one or more transgene protein products produced by the gene therapy vector(s). In various embodiments, the liposomes encapsulating one or more gene therapy vector antigens are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of liposomes encapsulating one or more transgene protein products produced by the gene therapy vector(s). In various embodiments, the liposomes encapsulating one or more gene therapy vector antigens are administered 1, 2, 3, 4, 5, 6, or 7 days prior to the administration of liposomes encapsulating one or more transgene protein products produced by the gene therapy vector(s). In various embodiments, the liposomes encapsulating one or more gene therapy vector antigens are administered one week, two weeks, three weeks, four weeks, 1 month, 2 months, 3 months, 4 months, 5 months, 6 months, 7 months, 8 months, 9 months, 10 months, 11 months, or 12 months prior to the administration of liposomes encapsulating one or more transgene protein products produced by the gene therapy vector(s).

In various embodiments, the liposomes encapsulating one or more gene therapy vector antigens are administered 5, 10, 15, 30, 45, or 60 minutes after the administration of liposomes encapsulating one or more transgene protein products produced by the gene therapy vector(s). In various embodiments, the liposomes encapsulating one or more gene therapy vector antigens are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of liposomes one or more encapsulating transgene protein products produced by the gene therapy vector(s). In various embodiments, the liposomes encapsulating one or more gene therapy vector antigens are administered 1, 2, 3, 4, 5, 6, or 7 days after the administration of liposomes encapsulating one or more transgene protein products produced by the gene therapy vector(s). In various embodiments, the liposomes encapsulating one or more gene therapy vector antigens are administered one week, two weeks, three weeks, four weeks, 1 month, 2 months, 3 months, 4 months, 5 months, 6 months, 7 months, 8 months, 9 months, 10 months, 11 months, or 12 months after the administration of liposomes encapsulating one or more transgene protein products produced by the gene therapy vector(s).

In various embodiments, the liposomes are administered intravenously, subcutaneously, intramuscularly, intraperitoneally, intranasally, or orally. In various embodiments, the liposomes are administered prior to, concomitantly, or after the administration of the gene therapy vector.

In various embodiments, the liposomes are administered 5, 10, 15, 30, 45, or 60 minutes prior to the administration of the gene therapy vector. In various embodiments, the liposomes are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours prior to the administration of the gene therapy vector. In various embodiments, the liposomes are administered 1, 2, 3, 4, 5, 6, or 7 days prior to the administration of the gene therapy vector. In various embodiments, the liposomes are administered one week, two weeks, three weeks, four weeks, 1 month, 2 months, 3 months, 4 months, 5 months, 6 months, 7 months, 8 months, 9 months, 10 months, 11 months, or 12 months prior to the administration of the gene therapy vector.

In various embodiments, the liposomes are administered 5, 10, 15, 30, 45, or 60 minutes after the administration of the gene therapy vector. In various embodiments, the liposomes are administered 1, 2, 3, 4, 5, 6, 7, 8, 9, 10, 11, 12, 13, 14, 15, 16, 17, 18, 19, 20, 21, 22, 23, 24, 36, 48, 72, or 96 hours after the administration of the gene therapy vector. In various embodiments, the liposomes are administered 1, 2, 3, 4, 5, 6, or 7 days after the administration of the gene therapy vector. In various embodiments, the liposomes are administered one week, two weeks, three weeks, four weeks, 1 month, 2 months, 3 months, 4 months, 5 months, 6 months, 7 months, 8 months, 9 months, 10 months, 11 months, or 12 months after to the administration of the gene therapy vector.

In various embodiments, administering the liposomes in a subject in need thereof induces antigen specific tolerance to the gene therapy vector antigen, portions thereof, or combinations thereof. In various embodiments, administering the liposomes in a subject in need thereof induces antigen specific tolerance to the transgene protein product, portions, or combinations thereof. In various embodiments, administering the liposomes in a subject reduces the inflammatory immune response to the gene therapy vector and/or the transgene protein product expressed by the vector. In various embodiments, administering the liposomes in a subject reduces the inflammatory immune response to one or more encapsulated antigens. In various embodiments, administering the liposomes in a subject in need thereof reduces the inflammatory response to the one or more antigens not encapsulated within the particle. Such an immune regulatory response has been referred to in the literature as ‘infectious tolerance’. In various embodiments, the inflammatory immune response is a humoral immune response and/or an adaptive immune response. In various embodiments, the immune response is an antibody response. In various embodiments, the antibody response is an IgA, IgG, IgE, or IgM response. In various embodiments, the antibody response is a neutralizing antibody response, for example the formation of neutralizing antibodies against the gene therapy vector antigen and/or the transgene protein produced by the vector. In various embodiments, the immune response is a T cell, B cell, NK cell monocyte, macrophage, eosinophil, or a basophil response.

In various embodiments, administering the liposomes in a subject in need thereof induces an antigen specific immune regulator response. In various embodiments, administering the liposomes in a subject induces regulatory T cells, B cells, monocytes, and/or macrophages. In various embodiments, administering TIMPs in a subject induces antigen specific Treg, Tr1, Mreg, and/or Breg cells.

Also contemplated are compositions described herein comprising a negatively charged particle encapsulating an antigen, wherein the antigen is one or more gene therapy vector antigens and/or one or more transgene protein products produced by the gene therapy vector(s), portions, or combinations thereof for use in inducing tolerance in a subject.

The disclosure also provides for use of a composition described herein comprising a negatively charged particle encapsulating an antigen, wherein the antigen is one or more gene therapy vector antigens and/or one or more transgene protein products produced by the gene therapy vector(s), portions, or combinations thereof in the preparation of a medicament for inducing tolerance in a subject.

In various embodiments, the negatively charged particle is a TIMP or a liposome as described herein.

It is understood that each feature or embodiment, or combination, described herein is a non-limiting, illustrative example of any of the aspects of the invention and, as such, is meant to be combinable with any other feature or embodiment, or combination, described herein. For example, where features are described with language such as “one embodiment”, “some embodiments”, “certain embodiments”, “further embodiment”, “specific exemplary embodiments”, and/or “another embodiment”, each of these types of embodiments is a non-limiting example of a feature that is intended to be combined with any other feature, or combination of features, described herein without having to list every possible combination. Such features or combinations of features apply to any of the aspects of the invention. Where examples of values falling within ranges are disclosed, any of these examples are contemplated as possible endpoints of a range, any and all numeric values between such endpoints are contemplated, and any and all combinations of upper and lower endpoints are envisioned.

The headings herein are for the convenience of the reader and not intended to be limiting. Additional aspects, embodiments, and variations of the invention will be apparent from the Detailed Description and/or Drawings and/or claims.

### BRIEF DESCRIPTION OF THE FIGURES

FIG. 1. Effect of CNP-GFP+CNP-VP1 treatment on GFP expression after initial administration of AAV8-GFP via intramuscular injection. Female BALB/c mice (6-8 weeks of age, n=10 per group) were injected intravenously with CNP-GFP (1.25 mg/mouse) and CNP-VP1 (1.25 mg/mouse) or Unloaded CNPs (Control) on Day −7 and Day 0. Mice were administered rAAV8-eGFP via intramuscular injection in the left quadricep on Day 0. Mice were monitored for transgene eGFP expression in the primed left quadricep on Day 0, 3, 7, 14, 21, and 28 by transcutaneous fluorescence spectroscopy. Treatment with CNP-GFP+CNP-VP1 led to significantly higher GFP expression when compared to Control (*p<0.05; ***p<0.001; ****p<0.0001).

FIG. 2A-2D. Effect of CNP-GFP+CNP-VP1 treatment on antigen specific CD8 T cells after initial administration and re-dosing of AAV8-GFP via intramuscular injection. Female BALB/c mice (6-8 weeks of age, n=10 per group) were injected intravenously with CNP-GFP (1.25 mg/mouse) and CNP-VP1 (1.25 mg/mouse) or Unloaded CNPs (Control) on Day −7 and Day 0. Mice were administered rAAV8-eGFP via intramuscular injection in the left quadricep on Day 0. On Day 28, mice were re-dosed with AAV8-GFP intramuscularly on the contralateral right quadricep (N=5 from each group). Seven days after re-dosing (Day 35), mice were sacrificed and both the right and left quadriceps were harvested for analysis. Frequencies and total cell counts of AAV8-VP1 and GFP specific CD8⁺ T cells were evaluated by flow cytometry using tetramer staining. Treatment with CNP-GFP+CNP-VP1 led to a significant reduction in the number of VP1 and GFP specific CD8+ T cells in both the initial tissue of AAV8-GFP administration (left quadricep) (FIG. 2A, 2B) and at the tissue of AAV8-GFP re-dosing (right quadricep) (FIG. 2C, 2D). (*p<0.05; **p<0.01).

FIG. 3. Effect of CNP-GFP+CNP-VP1 treatment on GFP expression after re-dosing of AAV8-GFP via intramuscular injection. Female BALB/c mice (6-8 weeks of age, n=10 per group) were injected intravenously with CNP-GFP (1.25 mg/mouse) and CNP-VP1 (1.25 mg/mouse) or Unloaded CNPs (Control) on Day −7 and Day 0. Mice were administered rAAV8-eGFP via intramuscular injection in the left quadricep on Day 0. On Day 35, the mice in each treatment group (N=5 per group) were re-dosed with AAV8-eGFP intramuscularly on the contralateral right quadricep and monitored for eGFP expression in the right quadricep by transcutaneous fluorescence spectroscopy. Mice treated with CNP-GFP+CNP-VP1 demonstrated significantly higher eGFP expression in the right quadricep on Days 14 and 21 after AAV8-GFP re-dosing when compared to Control (**p<0.01; ****p<0.0001). Relative to Control treatment, CNP-GFP+CNP-VP1 treatment led to a 58% increase in GFP expression from Day 14 to Day 21.

FIG. 4A-4C. Systemic administration of TIMPs encapsulating AAV8-GFP leads to tolerogenic phenotypes. C57BL/6 mice (age 6-8 weeks, n=5) were primed with either PBS or empty AAV8 capsid on day 0. Mice were administered 2 intravenous doses of CNP-GFP (1.25 mg/mouse)+CNP-VP1 (1.25 mg/mouse) on day 14 and day 21. Mice were challenged with AAV8-eGFP by IV administration on day 28. Mice were sacrificed and the final readout was taken on day 53. Mice treated with CNPs have decreased leukocytes in the hearts compared to control mice (FIG. 4A). CD4+ T cells show increased regulatory phenotypes in the spleen, liver, and heart (FIG. 4B). Mice treated with CNPs have increased regulatory T-cells (CD4+Foxp3+ and CD4+Ctla4+) in the spleen and liver when compared to control treated (PBS) mice (FIG. 4B). Mice receiving CNP therapy also had increased numbers of CD4+ cells expressing regulatory cytokine IL-10+ and CD44^{hi} memory marker in the spleen (FIG. 4B). Mice treated with CNPs have increased memory CD4+ T cells (CD44hi IFN-gamma−) and increased regulatory cells (CD4+Foxp3+, CD4+PD-1+, CD4+Ctla4+, CD4+IL-10+) (FIG. 4B) in the heart. CD8+ T cells have decreased effector phenotypes compared to in the heart of CNP treated mice (CD8+granzyme+, CD8+PD-1+, CD8+CD69+) (FIG. 4C). (*p<0.05; **p<0.01; ***p<0.001).

### DETAILED DESCRIPTION

Antigen-specific tolerance has been described as an attractive strategy to overcome the immunogenicity of gene therapy vectors and the protein therapeutics produced by the vectors. Several methods of inducing antigen-specific tolerance have been described but their translation into the clinic has been elusive¹⁶. There is a need for interventions that will allow safe administration of therapeutic viral vectors multiple times and overcome challenges associated with vector immunogenicity.

The present disclosure provides compositions of negatively charged particles encapsulating one or more gene therapy vectors, portions, or combinations thereof. The disclosure also provides compositions of negatively charged particles encapsulating one or more transgene protein products produced by gene therapy vectors, portions, or combinations thereof. Also included are methods of inducing antigen specific tolerance using the negatively charged particles described herein.

### Definitions

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

“Particle” as used herein refers to any non-tissue derived composition of matter, it may be a sphere or sphere-like entity, bead, or liposome. The term “particle”, the term “tolerizing immune modifying particle”, the term “carrier particle”, and the term “bead” may be used interchangeably depending on the context. Additionally, the term “particle” may be used to encompass beads and spheres.

“Negatively charged particle” as used herein refers to particles which have been modified to possess a net surface charge that is less than zero.

“Carboxylated particles” or “carboxylated beads” or “carboxylated spheres” includes any particle that has been modified to contain a carboxyl group on its surface. In some embodiments the addition of the carboxyl group enhances phagocyte/monocyte uptake of the particles from circulation, for instance through the interaction with scavenger receptors such as MARCO. Carboxylation of the particles can be achieved using any compound which adds carboxyl groups, including, but not limited to, poly (ethylene-maleic anhydride) (PEMA).

As used herein, the term “Th cell” or “helper T cell” refers to CD4⁺ cells. CD4⁺ T cells assist other white blood cells with immunologic processes, including maturation of B cells into plasma cells and memory B cells, and activation of cytotoxic T cells and macrophages. T cells become activated when they are presented with peptide antigens by MHC class Il molecules, which are expressed on the surface of antigen-presenting cells (APCs).

As used herein, the term “Th1 cell” refers to a subset of Th cells which produce pro-inflammatory mediators. Th1 cells secrete cytokines to facilitate immune response and play a role in host defense against pathogens in part by mediating the recruitment of neutrophils and macrophages to infected tissues. Th1 cells secrete cytokines including IFN-gamma, IL-2, IL-10, and TNF alpha/beta to coordinate defense against intracellular pathogens such as viruses and some bacteria.

As used herein, the term “Th2 cell” refers to a subset of Th cells that mediate the activation and maintenance of the antibody-mediated immune response against extracellular parasites, bacteria, allergens, and toxins. Th2 cells mediate these functions by producing various cytokines such as IL-4, IL-5, IL-6, IL-9, IL-13, and IL-17E (IL-25) that are responsible for antibody production, eosinophil activation, and inhibition of several macrophage functions, thus providing phagocyte-independent protective responses.

“Polypeptide” and “protein” refer to a polymer composed of amino acid residues, related naturally occurring structural variants, and synthetic non-naturally occurring analogs thereof, linked via peptide bonds or peptide bond isosteres. Synthetic polypeptides can be synthesized, for example, using an automated polypeptide synthesizer. The terms “polypeptide” and “protein” are not limited to a minimum length of the product. The term “protein” typically refers to large polypeptides. The term “peptide” typically refers to short polypeptides. Thus, peptides, oligopeptides, dimers, multimers, and the like, are included within the definition. Both full-length proteins and fragments thereof are encompassed by the definition. The terms “polypeptide” and “protein” also include post-expression modifications of the polypeptide or protein, for example, glycosylation, acetylation, phosphorylation and the like. Furthermore, for purposes of the present disclosure, a “polypeptide” can include “modifications,” such as deletions, additions, substitutions (which may be conservative in nature or may include substitutions with any of the 20 amino acids that are commonly present in human proteins, or any other naturally or non-naturally-occurring or atypical amino acids), and chemical modifications (e.g., addition of or substitution with peptidomimetics), to the native sequence. These modifications may be deliberate, as through site-directed mutagenesis, or through chemical modification of amino acids to remove or attach chemical moieties, or may be accidental, such as through mutations arising via hosts cells that produce the proteins or through errors due to PCR amplification prior to host cell transfection.

“Antigenic moiety” or “antigen” as used herein refers to any moiety, for example a peptide, that is recognized by the host's immune system. Examples of antigenic moieties include, but are not limited to, autoantigens, allergens, enzymes, and/or bacterial or viral proteins, peptides, drugs or components.

“Gene therapy vector antigen” as used herein refers to a gene therapy vector, or a portion or fragment thereof, e.g., a surface protein, that results in an immune reaction against the protein or portion or fragment thereof. A gene therapy vector antigen can include the whole gene therapy vector (e.g., viral, bacterial, bacteriophage, or other vector) or a protein part of the gene therapy vector, such as a viral capsid protein, envelope protein, or other protein or moiety associated with the vector that may elicit an immune response in a subject receiving the gene therapy vector.

“Pharmaceutically acceptable carrier” refers to any of the standard pharmaceutical carriers, buffers, and the like, such as a phosphate buffered saline solution, 5% aqueous solution of dextrose, and emulsions (e.g., an oil/water or water/oil emulsion). Non-limiting examples of excipients include adjuvants, binders, fillers, diluents, disintegrants, emulsifying agents, wetting agents, lubricants, glidants, sweetening agents, flavoring agents, and coloring agents. Suitable pharmaceutical carriers, excipients and diluents are described in Remington's Pharmaceutical Sciences, 19th Ed. (Mack Publishing Co., Easton, 1995). Preferred pharmaceutical carriers depend upon the intended mode of administration of the active agent. Typical modes of administration include enteral (e.g., oral) or parenteral (e.g., subcutaneous, intramuscular, intravenous or intraperitoneal injection; or topical, transdermal, or transmucosal administration) or via inhalation.

By “pharmaceutically acceptable” or “pharmacologically acceptable” is meant a material that is not biologically or otherwise undesirable, i.e., the material may be administered to an individual without causing any undesirable biological effects or without interacting in a deleterious manner with any of the components of the composition in which it is contained or with any components present on or in the body of the individual.

As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. The term does not denote a particular age or gender.

The term “epitope” refers to that portion of any molecule capable of being recognized by and bound by a selective binding agent at one or more of the antigen binding regions. Epitopes usually consist of chemically active surface groupings of molecules, such as, amino acids or carbohydrate side chains, and have specific three-dimensional structural characteristics as well as specific charge characteristics. Epitopes as used herein may be contiguous or non-contiguous. Moreover, epitopes may be mimetic (mimotopes) in that they comprise a three-dimensional structure that is identical to the epitope used to generate the antibody, yet comprise none or only some of the amino acid residues found in the target that were used to stimulate the antibody immune response. As used herein, a mimotope is not considered a different antigen from the epitope bound by the selective binding agent; the selective binding agent recognizes the same three-dimensional structure of the epitope and mimotope.

The term “therapeutically effective amount” is used herein to indicate the amount of antigen-specific composition of the disclosure that is effective to ameliorate or lessen one or more symptoms or signs of disease to be treated.

The terms “treat”, “treated”, “treating” and “treatment”, as used with respect to methods herein refer to eliminating, reducing, suppressing or ameliorating, either temporarily or permanently, either partially or completely, a clinical symptom, manifestation or progression of an event, disease or condition. Such treating need not be absolute to be useful.

### Particles

Tolerizing Immune Modifying Particles (TIMPs), comprising one or more antigens, have been previously described for the induction of antigen specific tolerance for treating inflammatory conditions (e.g., autoimmune diseases and allergies) (WO20131319253 and WO2015023796 incorporated herein by reference). In several preclinical models of autoimmune diseases and allergies, TIMPs have demonstrated efficacy at inducing antigen specific tolerance and inhibition of pathologic inflammatory immune responses.

The size and charge of the particles are important for tolerance induction. While the particles will differ in size and charge based on the antigen encapsulated within them, in general, particles described herein are effective at inducing tolerance when they are between about 100 nanometers and about 1500 nanometers and have a charge of between 0 to about −100 mV. In various embodiments, the particles are 400-800 nanometers in diameter and have a charge of between about −25 mV and −70 mV. In various embodiments, the particles are 400-800 nanometers in diameter and have a charge of between about −30 mV and −80 mV. In various embodiments, the particles are 400-800 nanometers in diameter and have a charge of between about −30 mV and −60 mV. The average particle size and charge of the particles can be slightly altered in the lyophilization process, therefore, both post-synthesis averages and post-lyophilization averages are described. As used herein, the term “post-synthesis size” and “post synthesis charge” refer to the size and charge of the particle prior to lyophilization. The term “post lyophilization size” and “post lyophilization charge” refer to the size and charge of the particle after lyophilization.

In some embodiments, the particle is non-metallic. In these embodiments the particle may be formed from a polymer. In a preferred embodiment, the particle is biodegradable in an individual. In this embodiment, the particles can be provided in an individual across multiple doses without there being an accumulation of particles in the individual. Examples of suitable particles include polystyrene particles, PLGA particles, PLURONICS stabilized polypropylene sulfide particles, and diamond particles.

Preferably the particle surface is composed of a material that minimizes non-specific or unwanted biological interactions. Interactions between the particle surface and the interstitium may be a factor that plays a role in lymphatic uptake. The particle surface may be coated with a material to prevent or decrease non-specific interactions. Steric stabilization by coating particles with hydrophilic layers such as poly(ethylene glycol) (PEG) and its copolymers such as PLURONICS® (including copolymers of poly(ethylene glycol)-bl-poly(propylene glycol)-bl-poly(ethylene glycol)) may reduce the non-specific interactions with proteins of the interstitium as demonstrated by improved lymphatic uptake following subcutaneous injections. All of these facts suggest relevance of the physical properties of the particles in terms of lymphatic uptake. Biodegradable polymers may be used to make all or some of the polymers and/or particles and/or layers. Biodegradable polymers may undergo degradation, for example, by a result of functional groups reacting with the water in the solution. The term “degradation” as used herein refers to becoming soluble, either by reduction of molecular weight or by conversion of hydrophobic groups to hydrophilic groups. Polymers with ester groups are generally subject to spontaneous hydrolysis, e.g., polylactides and polyglycolides.

Particles disclosed herein may also contain additional components. For example, carriers may have imaging agents incorporated or conjugated to the carrier. An example of a carrier nanosphere having an imaging agent that is currently commercially available is the Kodak X-sight nanospheres. Inorganic quantum-confined luminescent nanocrystals, known as quantum dots (QDs), have emerged as ideal donors in FRET applications: their high quantum yield and tunable size-dependent Stokes Shifts permit different sizes to emit from blue to infrared when excited at a single ultraviolet wavelength. (Bruchez, et al., Science, 1998, 281, 2013; Niemeyer, C. M Angew. Chem. Int. Ed. 2003, 42, 5796; Waggoner, A. Methods Enzymol. 1995, 246, 362; Brus, L. E. J. Chem. Phys. 1993, 79, 5566). Quantum dots, such as hybrid organic/inorganic quantum dots based on a class of polymers known as dendrimers, may be used in biological labeling, imaging, and optical biosensing systems. (Lemon, et al., J. Am. Chem. Soc. 2000, 122, 12886). Unlike the traditional synthesis of inorganic quantum dots, the synthesis of these hybrid quantum dot nanoparticles does not require high temperatures or highly toxic, unstable reagents. (Etienne, et al., Appl. Phys. Lett. 87, 181913, 2005).

Particles can be formed from a wide range of materials. The particle is preferably composed of a material suitable for biological use. For example, particles may be composed of glass, silica, polyesters of hydroxy carboxylic acids, polyanhydrides of dicarboxylic acids, or copolymers of hydroxy carboxylic acids and dicarboxylic acids. More generally, the carrier particles may be composed of polyesters of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy hydroxy acids, or polyanhydrides of straight chain or branched, substituted or unsubstituted, saturated or unsaturated, linear or cross-linked, alkanyl, haloalkyl, thioalkyl, aminoalkyl, aryl, aralkyl, alkenyl, aralkenyl, heteroaryl, or alkoxy dicarboxylic acids. Additionally, carrier particles can be quantum dots, or composed of quantum dots, such as quantum dot polystyrene particles (Joumaa et al. (2006) Langmuir 22: 1810-6). Carrier particles including mixtures of ester and anhydride bonds (e.g., copolymers of glycolic and sebacic acid) may also be employed. For example, carrier particles may comprise materials including polyglycolic acid polymers (PGA), polylactic acid polymers (PLA), polysebacic acid polymers (PSA), poly(lactic-co-glycolic) acid copolymers (PLGA or PLG; the terms are interchangeable), poly(lactic-co-sebacic) acid copolymers (PLSA), poly(glycolic-co-sebacic) acid copolymers (PGSA), polypropylene sulfide polymers, poly(caprolactone), chitosan, etc. Other biocompatible, biodegradable polymers useful in the present invention include polymers or copolymers of caprolactones, carbonates, amides, amino acids, orthoesters, acetals, cyanoacrylates and degradable urethanes, as well as copolymers of these with straight chain or branched, substituted or unsubstituted, alkanyl, haloalkyl, thioalkyl, aminoalkyl, alkenyl, or aromatic hydroxy- or di-carboxylic acids. In addition, the biologically important amino acids with reactive side chain groups, such as lysine, arginine, aspartic acid, glutamic acid, serine, threonine, tyrosine and cysteine, or their enantiomers, may be included in copolymers with any of the aforementioned materials to provide reactive groups for conjugating to antigen peptides and proteins or conjugating moieties. Biodegradable materials suitable for the present invention include diamond, PLA, PGA, polypropylene sulfide, and PLGA polymers. Biocompatible but non-biodegradable materials may also be used in the carrier particles of the invention. For example, non-biodegradable polymers of acrylates, ethylene-vinyl acetates, acyl substituted cellulose acetates, non-degradable urethanes, styrenes, vinyl chlorides, vinyl fluorides, vinyl imidazoles, chlorosulphonated olefins, ethylene oxide, vinyl alcohols, TEFLON® (DuPont, Wilmington, Del.), and nylons may be employed.

In certain embodiments, the particle is a co-polymer having a molar ratio from about 80:20 to about 100:0. Suitable co-polymer ratio of present immune modified particles may be 25:75, 30:70, 35:65, 40:60, 45:55, 50:50, 55:45, 60:40, 65:35, 70:30, 75:25, 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, 99:1, or 100:0. In various embodiments, the particle is a PLURONICS stabilized polypropylene sulfide particle, a polyglycolic acid particle (PGA), a polylactic acid particle (PLA), or a poly(lactic-co-glycolic acid) particle. In various embodiments, the particle is a carboxylated PLGA particle. In various embodiments, the particle has a copolymer ratio of polylactic acid/polyglycolic acid 80:20: polylactic acid/polyglycolic acid 90:10: or polylactic acid: polyglycolic acid/50:50. In various embodiments, the particle is a poly(lactic-co-glycolic acid) particle and has a copolymer ratio of about 50:50 polylactic acid:polyglycolic acid. In various embodiments, the particle comprises about 50:50, about 80:20 to about 100:0 polylactic acid: polyglycolic acid or from about 50:50, about 80:20 to about 100:0 polyglycolic acid: polylactic acid. In various embodiments, the particle comprises 50:50 polylactic acid: polyglycolic acid. In various embodiments, the particle comprises polylactic acid: polyglycolic acid from about 99:1 to about 1:99, e.g., about 99:1, about 95:5, about 90:10, about 85:15, about 80:20, about 75:25, about 70:30, about 65:35, about 60:40, about 55:45, about 50:50, about 45:55, about 40:60, about 35:65, about 30:70, about 25:75, about 20:80, about 15:85, about 10:90, about 5:95, and about 1:99, including all values and ranges that lie in between these values.

It is contemplated that the particle may further comprise a surfactant. The surfactant can be anionic, cationic, or nonionic. Surfactants in the poloxamer and poloaxamines family are commonly used in particle synthesis. Surfactants that may be used, include, but are not limited to PEG, Tween-80, gelatin, dextran, pluronic L-63, polyvinyl alcohol (PVA), polyacrylic acid (PAA), methylcellulose, lecithin, didodecyldimethylammonium bromide (DMAB) and poly(ethylene-alt-maleic acid) (PEMA). Additionally, biodegradable and biocompatible surfactants including, but not limited to, vitamin E TPGS (D-α-tocopheryl polyethylene glycol 1000 succinate), poly amino acids (e.g., polymers of lysine, arginine, aspartic acid, glutamic acid, serine, threonine, tyrosine and cysteine, or their enantiomers), and sulfate polymers. In some embodiments, two surfactants are used. For example, if the particle is produced by a double emulsion method, the two surfactants can include a hydrophobic surfactant for the first emulsion, and a hydrophobic surfactant for the second emulsion.

In various embodiments, the polypeptide antigens are encapsulated in the particles by a single-emulsion process. In a further embodiment, the polypeptide antigens are more hydrophobic. Sometimes, the double emulsion process leads to the formation of large particles which may result in the leakage of the hydrophilic active component and low entrapment efficiencies. The coalescence and Ostwald ripening are two mechanisms that may destabilize the double-emulsion droplet, and the diffusion through the organic phase of the hydrophilic active component is the main mechanism responsible of low levels of entrapped active component. In some embodiments, it may be beneficial to reduce the nanoparticle size. One strategy to accomplish this is to apply a second strong shear rate. The leakage effect can be reduced by using a high polymer concentration and a high polymer molecular mass, accompanied by an increase in the viscosity of the inner water phase and in increase in the surfactant molecular mass. In certain embodiments, the particles encapsulating antigens are manufactured by nanoprecipitation, co-precipitation, inert gas condensation, sputtering, microemulsion, sol-gel method, layer-by-layer technique or ionic gelation method. Several methods for manufacturing nanoparticles have been described in the literature and are incorporated herein by reference^{17,18}.

In some embodiments, the particle is a liposome. Liposomes may be prepared from a variety of lipid materials including, but not limited to, lipids of phosphatidyl choline, phosphatidyl serine, phosphatidyl inositol, phosphatidyl glycerol, phosphatidyl ethanolamine, phosphatidic acid, dicetyl phosphate, monosialoganglioside, polyethylene glycol, stearyl armine, ovolecithin and cholesterol, as well as mixtures of these in varying stoichiometries. Liposomes, as used herein, may also be formed from non-lipid amphipathic molecules, such as block copolymers of poly(oxyethylene-b-isoprene-b-oxye-thylene) and the like. In preferred embodiments, the liposomes are prepared from lipids or incorporate lipids that will form negatively charged liposomes, such as those produced from phosphatidyl serine, dicetyl phosphate, and dimyristoyl phosphatidic acid. In various embodiments, the negatively charged liposomes have a zeta potential from about −100 mV to about 0 mV, from about −100 mV to about −25 mV, from about −100 to about −30 mV, from about −80 mV to about −30 mV, from about −75 mV to about −30 mV, from about −70 mV to about −30 mV, from about −75 to about −35 mV, from about −70 to about −25 mV, from about −60 mV to about −30 mV, from about −60 mV to about −35 mV, or from about −50 mV to about −30 mV. In various embodiments, the zeta potential is about −25 mV, −30 mV, −35 mV, −40 mV, −45 mV, −50 mV, −55 mV, −60 mV, −65 mV, −70 mV, −75 mV, −80 mV, −85 mV, −90 mV, −95 mV or −100 mV, including all values and ranges therein. In various embodiments, the liposomes have a negative zeta potential of between −30 mV to −80 mV. In various embodiments, the liposomes have a negative zeta potential of between −30 mV to −60 mV.

In various embodiments, the liposomes encapsulate one or more gene therapy vector antigens and/or a portion thereof, or combinations of gene therapy vector antigens or portions thereof. In various embodiments, the liposomes encapsulate a therapeutic protein and/or a fragment or portion thereof produced by the gene therapy vector. In various embodiments, the therapeutic protein produced by the gene therapy vector is a protein, a polypeptide, or a peptide. In various embodiments, the therapeutic protein is a cytokine, a chemokine, a hormone, a growth factor, an enzyme, or an antibody. In various embodiments, the gene therapy vector antigen is selected from the group consisting of adenovirus, adeno-associated virus (AAV), AAV-1, AAV-2, AAV-3, AAV-4, AAV-5, AAV-6, AAV-7, AAV-8, AAV-9, AAV-10, AAV-12, Anc80, synthetic AAV, combinations or engineered versions thereof, AAV capsid protein VP1, AAV capsid protein VP2, AAV capsid protein VP3, herpes simplex virus, hepatitis B virus, lentivirus, retrovirus, alphavirus, flavivirus, rhabdovirus, measles virus, Newcastle disease virus, Coxsackieviruse, transfusion transmitted viruses, anellovirus, human papilloma virus, poxvirus, vaccinia virus, modified Ankara virus, vesicular stomatitis virus, picornavirus, tobacco mosaic virus, cowpea mosaic virus, cowpea chlorotic mottle virus, physalis mosaic virus, Red clover necrotic mosaic virus, potato virus x, comovirus, chicken anemia virus, cucumber mosaic virus, oncolytic virus, chimeric virus, a synthetic virus, a mosaic virus, or a pseudotyped virus, bacteria, bacteriophage, yeast, exosome, or erythrocyte.

In various embodiments, the liposomes encapsulate one or more gene therapy vector antigens, wherein the size of liposomes is between 100 and 1000 nm in diameter, and the liposomes have a negative zeta potential between −100 mV and −0 mV. In various embodiments, the liposomes encapsulate one or more gene therapy vector antigens, portions thereof or combinations thereof, wherein the size of the liposomes is between 400 and 800 nm and the liposomes have a negative zeta potential between −30 mV and −80 mV.

In various embodiments, the liposomes encapsulate a therapeutic protein and/or a fragment or portion thereof produced by the gene therapy vector, wherein the size of the liposomes is between 100 and 1000 nm, and the liposomes have a negative zeta potential between −100 mV and 0 mV. In various embodiments, the liposomes encapsulate a therapeutic protein and/or a fragment or portion thereof produced by the gene therapy vector, wherein the size of the liposomes is between 400 and 800 nm and the liposomes have a negative zeta potential between −30 mV and −80 mV. In various embodiments, the liposomes are used to induce tolerance in a subject in need thereof. In various embodiments, the liposome administration is intravenous.

### Gene Therapy Vectors

Gene therapy vectors are designed to deliver genes encoding therapeutic products to a subject in order to alleviate symptoms of disease, such as a genetic disorder or cancer. Gene therapy vectors include virus, bacteria, bacteriophage, yeast, erythrocyte, or exosome vectors.

Viral vectors commonly used for human gene therapy include adenovirus, adeno-associated virus (AAV), and retrovirus, such as lentivirus.

Adenovirus vectors are frequently derived from human serotypes HAd2, HAd5, HAd26 and HAd35, or engineered, such as “high-capacity” adenoviral vectors (HCAds) or oncolytic vectors, and can also be derived from non-human mammalian Ad viruses (Bulcha et al., Signal Transduction and Targeted Therapy volume 6, Article number: 53 (2021)). Adenovirus expression can induce immune response to early transcription genes E1 (E1A, E1B), E2, E3, and E4, hexons, which are the most abundant structural components on the capsid surface capsid and 12 penton proteins located on the capsid vertices and give rise to the extending fibers found on these viruses. Carrier particles comprising these viral antigens are contemplated for inducing tolerance to these gene therapy vector antigens.

AAV vectors are small viral vectors having an icosahedral capsid composed primarily of three proteins, VP1, VP2 and VP3. Twelve serotypes of AAV are known. (Bulcha et al., supra). Commonly used serotypes for gene therapy include AAV-1, AAV-2, AAV-5, AAV-9, Anc80, while the other serotypes include AAV-3, AAV-4, AAV-6, AAV-7, AAV-8, AAV-10, AAV-12, and synthetic AAVs. Native and engineered capsid proteins have been used in generating AAV vectors for administration in gene therapy. Carrier particles comprising wild type or engineered AAV viral capsid proteins are contemplated herein for inducing tolerance to these gene therapy vector antigens.

Lentiviruses are enveloped viruses having three primary gene sets, gag, pol and env genes, and can have auxiliary genes useful to improve proliferation and pathogenesis (tat, rev, vif, vpr, vpu, and nef). Gag genes encode for the structural envelope proteins. However, engineered lentiviral vectors may contain a VSV-G protein in place of the envelope proteins. (Bulcha et al., supra). Carrier particles comprising wild type or engineered lentiviral proteins are contemplated herein for inducing tolerance to these gene therapy vector antigens.

Bacterial vectors have been generated to deliver a gene or protein to target cancer cells (Baban et al., Bioengineered Bugs 1:6, 385-394, 2010), and include vectors derived from Salmonella, Shigella, Listeria, or E. coli. These vectors have been used to express human tumor antigens, cytokines, growth factors, enzymes, and therapeutic proteins. Another use of a bacterial vector is in bactofection, which refers to bacterial transfer of bacteriophage or plasmid DNA to mammalian cells. Carrier particles comprising bacterial or plasmid antigens are contemplated herein for inducing tolerance to these gene therapy vector antigens.

Yeast gene therapy vectors expressing tumor or HIV antigens have been shown to induce tumor reactive responses in vivo.²¹ Exosomes and polyethyleneimine matrix (EPM) have been used to administer short interfering RNA (siRNA) or plasmid DNA (pDNA) in patients and exosomes have been used to deliver CRISPR/Cas9 plasmids to cancer cells.^{22,23} Erythrocytes, e.g., erythrocyte ghosts, have been shown to effectively deliver plasmid DNA to cells^{24,} or can be loaded with proteins for delivery in vivo.²⁵

### Antigens

An antigen refers to a discreet portion of a molecule, such as a polypeptide or peptide sequence, a 3-D structural formation of a polypeptide or peptide, a polysaccharide or polynucleotide that can be recognized by an host immune cell. Antigen-specific refers to the ability of a subject's host cells to recognize and generate an immune response against an antigen alone, or to molecules that closely resemble the antigen, as with an epitope or mimotope.

“Anergy,” “tolerance,” or “antigen-specific tolerance” refers to insensitivity of T cells to T cell receptor-mediated stimulation. Such insensitivity is generally antigen-specific and persists after exposure to the antigenic peptide has ceased. For example, anergy in T cells is characterized by lack of cytokine production, e.g., IL-2. T-cell anergy occurs when T cells are exposed to antigen and receive a first signal (a T cell receptor or CD-3 mediated signal) in the absence of a second signal (a costimulatory signal). Under these conditions, re-exposure of the cells to the same antigen (even if re-exposure occurs in the presence of a costimulatory molecule) results in failure to produce cytokines and subsequently failure to proliferate. Thus, a failure to produce cytokines prevents proliferation. Anergic T cells can, however, proliferate if cultured with cytokines (e.g., IL-2).

It is contemplated that the tolerizing therapy described herein is antigen-specific. For example, TIMPs administered as tolerizing therapy encapsulate one or more antigens associated with said tolerizing therapy and associated disease or condition being treated. It is contemplated that the TIMPs used in tolerizing therapy comprise one or more gene therapy vector antigens, portions thereof, or combinations thereof, and/or transgene protein products produced by the gene therapy vector, portions thereof, or combinations thereof.

Exemplary gene therapy vectors include, but are not limited to, adenovirus, adeno-associated virus (AAV), herpes simplex virus, hepatitis B virus, lentivirus, retrovirus, alphavirus, flavivirus, rhabdovirus, measles virus, Newcastle disease virus, Coxsackievirus, transfusion transmitted viruses, anellovirus, human papilloma virus, poxvirus, vaccinia virus, modified Ankara virus, vesicular stomatitis virus, picornavirus, tobacco mosaic virus, cowpea mosaic virus, cowpea chlorotic mottle virus, physalis mosaic virus, Red clover necrotic mosaic virus, potato virus x, comovirus, chicken anemia virus, cucumber mosaic virus, AAV-1, AAV-2, AAV-3, AAV-4, AAV-5, AAV-6, AAV-7, AAV-8, AAV-9, AAV-10, AAV-12, Anc80, and a synthetic AAV. It is contemplated that the particles may encapsulate viral vector capsid or envelope proteins, portions or fragments thereof, or combinations thereof. Capsid proteins include, e.g., AAV-Po1 VP1 (UNIPROT ID: C0LA97), AAV-Po1 VP2 (UNIPROT ID: C0LA98), AAV-Po1 VP3 (UNIPROT ID: COLA99), AAV-Po2 VP1 (UNIPROT ID: C0LAA0), AAV-Po3 (UNIPROT ID: C0LA95), AAV VP1 (UNIPROT ID: B4Y875), AAV-1 capsid protein (UNIPROT ID: Q9WBP8), AAV-2 VP1 (UNIPROT ID: P03135), AAV-3 capsid protein (UNIPROT ID: Q65311), AAV-3B VP1 (UNIPROT ID: O56139), AAV-4 capsid (UNIPROT ID: O41855), AAV6 VP1 (UNIPROT ID: O56137), AAV7 capsid protein (UNIPROT ID: Q8JQG0), AAV8 capsid protein (UNIPROT ID: Q8JQF8), AAV9 VP1 (UNIPROT ID: Q6JC40), AAV-10 capsid protein (UNIPROT ID: Q5Y9B4), AAV-11 capsid protein (UNIPROT ID: Q5Y9B2), AAV13 capsid protein (UNIPROT ID: B5SUY7), and AAV12 VP1 (UNIPROT ID: A9RAI0).

In certain embodiments, one, two, three, or a higher number of antigens or antigenic peptides are used in the TIMPs. In certain embodiments, the one or more antigens are encapsulated in the TIMP by covalent linkage to the interior surface of the particle (See e.g., US Patent Publication US20190282707, herein incorporated by reference). In certain embodiments, it is contemplated that sequences of two or more antigens are linked in a fusion protein and encapsulated within a TIMP described herein. Methods for making TIMPs with linked epitopes are described in US Patent Publication US20190365656, herein incorporated by reference.

Exemplary enzymes useful for treating enzymatic diseases and produced by a gene therapy vector include imiglucerase, taliglucerase alfa, velaglucerase alfa, β-glucocerebrosidase, alglucerase, agalsidase beta, agalsidase alpha, sebelipase alpha, alpha-L-iduronidase, human iduronate-2-sulfatase, N-sulphoglucosamine sulphohydrolase, elosulfase alpha, galsulfase, alpha-glucosidase (GAA), human alpha mannosidase, Factor VIII, Factor IX, beta-galactosidase, arginase, dystrophia myotonica-protein kinase, ornithine transcarbamylase, NADPH oxidase, NADH dehydrogenase 4, adenosine deaminase, lipoprotein lipase, beta-glucocerebrosidase, myotubularin, arylsulfatase A, DOPA decarboxylase, matrix metallopeptidase 1, fumaryl acetoacetate hydrolase, phenylalanine hydroxylase, pyruvate kinase, porphobilinogen deaminase, alkaline phosphatase, sucrase-isomaltase, acid sphingomyelinease, heparan sulfase sulfatase, N-acetylgalactosamine-6-sulfatase, N-acetylgalactosamine-4-sulfatase, Alpha-1 proteinase inhibitor, 1-esterase inhibitor, fibrinogen, Factor VIIa, Factor X, Factor XI, Factor XII, Protein C, anti-thrombin III, ectonucleotide pyrophosphatase/phosphodiesterase 1, ectonucleotide pyrophosphatase/phosphodiesterase 3, and aromatic L-amino acid decarboxylase.

In certain embodiments, the protein therapeutic is an antibody, e.g., a monoclonal. It is also contemplated that the antibody is mono-specific, bi-specific, tri-specific, or bi-specific T-cell engager. In various embodiments the antibody targets receptor tyrosine kinase (RTK), EGFR, VEGF, VEGFR, PDGF, PDGFR, HER2/Neu, ER, PR, TGF-β1, TGF-β2, TGF-β3, SIRP-α, PD-1, PD-L1, CTLA-4, CD3, CD25, CD19, CD20, CD39, CD47, CD73, FAP, IL-1β, IL-12, IL-2R, IL-15, IL-15R, IL-23, IL-33, IL-2R, IL-4Rα, T-cells, B-cells, NK cells, macrophages, monocytes, and/or neutrophils. In various embodiments, the antibody is selected from the group consisting of abciximab, adalimumab, alemtuzumab, avelumab, azetolizumab, basiliximab, bevacizumab, bezlotoxumab, blinatumomab, canakinumab, certolizumab, cetuximab, daclizumab, denosumab, durvalumab, efalizumab, emicizumab, etokimab, golimumab, ipilimumab, ixekizumab, infliximab, natalizumab, nivolumab, olaratumab, omalizumab, ofatimumab, palivizumab, panitumumab, pembrolizumab, ramucirumab, rituximab, tocilizumab, trastuzumab, tremelimumab, secukinumab, ustekinumab, and vedolizumab.

### Methods of Use

Provided herein is a method of inducing tolerance to a subject in need thereof comprising administering in a subject a composition comprising negatively charged particles encapsulating an antigen, wherein the antigen is one or more gene therapy vector antigens and/or one or more transgene protein products produced by the gene therapy vector(s), portions, or combinations thereof.

In various embodiments, the subject is a subject who has received gene therapy, a subject who is receiving gene therapy, or a subject who will receive gene therapy. In various embodiments, the subject has received one treatment of gene therapy and may undergo another gene therapy treatment regimen, i.e., redosing of gene therapy.

In various embodiments, the subject is receiving gene therapy to treat cancer, autoimmune disease, allergy, cardiovascular disease, metabolic disease, enzyme deficiency, or protein deficiency.

In various embodiments, the subject is suffering from a rare and inherited genetic disorders, and receiving gene therapy to provide a transgene protein product produced by the vector which is an enzyme. In various embodiments, the subject is suffering from a rare and inherited genetic disorders selected form the group consisting of Hemophilia A, Hemophilia B, lysosomal storage diseases, Fabry disease, Gaucher disease, Pompe disease, Niemann-Pick disease, Tay-Sachs disease, macular degeneration, mucopolysaccharidosis, venous thrombosis, von Willebrand disease, purpura fulminans, growth-hormone deficiency, gangliosidosis, alkaline hypophosphatasia, cholesterol ester storage disease, hyperuricemia, Duchenne Muscular Dystrophy, Huntington's disease, Parkinson's, Alzheimer's disease, choroiderimia, Stargardt Disease, Batten disease, spinocerebellar ataxia, ALS, frontotemporal lobar degeneration, ornithine transcarbamylase deficiency, retinitis pigmentosa, RPE-65 mutation-associated diseases, epidermolysis bullosa, recessive dystrophic epidermolysis bullosa, spinal muscular atrophy, phenylketonuria (PKU), X-linked myotubular myopathy, Crigler-Najjar syndrome, catecholaminergic polymorphic ventricular tachycardia, glycogen storage disease type 1, alpha-mannosidosis, Fragile X-syndrome, arginase deficiency, X-linked chronic granulomatous disease, adenosine deaminase deficiency, Leber's congenital amaurosis, lipoprotein lipase deficiency, cerebral adrenoleukodystrophy, metachromatic leukodystrophy, Fanconi anemia, achromatopsia, scleroderma, osteogenesis imperfecta, coronary artery disease, tyrosinemia, peripheral neuropathy, optic neuropathy, coronary artey disease, respiratory syncytial virus (RSV)-mediated lower respiratory tract disease, Danon disease, severe leukocyte adhesion deficiency, pyruvate kinase deficiency, Charcot Marie Tooth disease, Wiskott Aldrich syndrome, alpha-synuclein tauopathies, refractory angina due to myocardial ischemia, myotonic dystrophy type I, claudication, peripheral artery disease, methylmalonic acidemia, MPS I, MPS II, MPS III, MPS IV, MPS VI, MPS VII, MPS IX, calcification/ossification disorders, ENPP1 deficiency, ENPP3 deficiency, ABCC6 deficiency, aromatic L-amino acid decarboxylase deficiency, Angelman syndrome, hyperphenylalaninaemia, dementia, Rett syndrome and Usher syndrome.

In various embodiments, the autoimmune disease is selected from the group consisting of multiple sclerosis, Addison's disease, ankylosing spondylitis, alopecia, osteoarthritis, psoriatic arthritis, scleroderma, type-I diabetes, rheumatoid arthritis, thyroiditis, systemic lupus erythematosus, Reynaud's syndrome, Behcets syndrome, Sjorgen's syndrome, autoimmune uveitis, Eaton Lamberts disease, autoimmune myocarditis, inflammatory bowel disease, amyotrophic lateral sclerosis (ALS), systemic lupus erythematosus, neuromyelitis optica, idiopathic thrombocytopenia purpura, thrombotic thrombocytopenia purpura, membranous nephropathy, bullous phemphigoid, phemphigus vulgaris, myasthenia gravis, Celiac disease, ulcerative colitis, Crohn's disease, erythema nodosa, glomerulonephritis, Goodpasture's syndrome, granulomatosis, Grave's disease, Guillain-Barre syndrome, Hashimoto disease, hemolytic anemia, Kawasaki Disease, mixed connective tissue disease, multifocal motor neuropathy, peripheral biliary cirrhosis, polyangiitis overlap syndrome, scleroderma type 1, sclerosis cholangitis, Stiffman syndrome, Takayasu arteritis, vitiligo or Wegeners granulomatosis.

In various embodiments, the allergy is a food allergy. In various embodiments, the allergy is an environmental allergy. In various embodiments, the food allergy is a peanut allergy, tree nut allergy, milk allergy, egg allergy, fish allergy, wheat allergy, celery allergy, or peach allergy. In various embodiments, the environmental allergy is pollen allergy, dust allergy, pet dander allergy, or mold allergy. In various embodiments, the pet allergy is cat allergy or dog allergy.

In various embodiments, the cancer is selected from the group consisting of brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, testicular cancer, leukemia, lymphoma, melanoma and mesothelioma.

In various embodiments, the carrier particles comprise one or more gene therapy vector antigens. In various embodiments, the carrier particles comprise one or more transgene protein products produced by the gene therapy vector(s), portions thereof, combinations thereof, or one or more antigenic epitopes thereof. In various embodiments, the carrier particles comprise both one or more gene therapy vector antigens and a transgene protein product or one or more antigenic epitopes thereof.

If carrier particles comprising a single antigen are administered in combination with another carrier particle comprising a different antigen, or second agent, the particles and or second agent can be administered concurrently or sequentially. Concomitant or concurrent administration of two therapeutic agents does not require that the agents be administered at the same time or by the same route, as long as there is an overlap in the time period during which the agents are exerting their therapeutic effect. Simultaneous or sequential administration is contemplated, as is administration on different days or weeks. It is further contemplated that the therapeutics are administered in a separate formulation and administered concurrently or concomitantly, with concurrently referring to agents given within 30 minutes of each other. Prior administration refers to administration of a therapeutic within the range of one week prior to treatment with a carrier particle, up to 30 minutes before administration of a carrier particle. Subsequent administration is meant to describe administration from 30 minutes after treatment up to one week after administration

In various embodiments, a carrier particle is administered at a dose from about 0.001 to about 10 mg/kg, from about 0.005 to about 12 mg/kg, from about 0.01 to about 12 mg/kg, from about 0.05 to about 12 mg/kg, from about 0.1 to about 12 mg/kg, about 0.5 to 10 mg/kg, from about 1 to 8 mg/kg, from about 1.5 to 10 mg/kg, from about 2 to 12 mg/kg, from about 2 to 10 mg/kg, from about 3 to 10 mg/kg, from about 4 to 10 mg/kg, from about 4 to 12 mg/kg, or from about 5 to 12 mg/kg. Optionally, the carrier particle is administered in a dose of about 0.001 mg/kg, about 0.0025 mg/kg, about 0.005 mg/kg, about 0.01 mg/kg, about 0.025 mg/kg, about 0.05 mg/kg, 0.1 mg/kg, 0.25, 0.5 mg/kg, 1.0 mg/kg, 2.0 mg/kg, 4.0 mg/kg, 6 mg/kg, 8.0 mg/kg, 10 mg/kg, or 12 mg/kg. Alternatively, the carrier particle is administered at a dose of about 0.1 mg, 0.25 mg, 0.5 mg, 1 mg, 2 mg, 2.5 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, or 800 mg. In another embodiment, carrier particles are administered at a concentration of between about 0.0005 mg/mL and about 50 mg/mL between about 0.05 mg/mL and about 50 mg/mL, optionally about 0.0005 mg/mL, 0.001 mg/mL, 0.005 mg/mL, 0.01 mg/mL, 0.05 mg/mL, 0.1 mg/mL, 0.5 mg/mL, 1 mg/mL, 2 mg/mL, 3 mg/mL, 4 mg/mL, 5 mg/mL, 6 mg/mL, 7 mg/mL, 8 mg/mL, 9 mg/mL, 10 mg/mL, 11 mg/mL, 12.5 mg/mL, 15 mg/mL, 17.5 mg/mL, 20 mg/mL, 25 mg/mL, 30 mg/mL, 40 mg/mL, or 50 mg/mL.

In some embodiments, the carrier particles comprising one or more gene therapy vector antigens and/or one or more transgene protein products produced by the gene therapy vectors are administered in combination with one or more therapeutics. In various embodiments the therapeutic is adoptive transfer of Tregs, CAR Tregs, TCR Tregs. In various embodiments the therapeutic blocks or inhibits innate immune responses, complement responses, B cell responses, and/or T cell responses against the gene therapy vector and/or transgene protein produced by the gene therapy vector.

In various embodiments, the therapeutic is an FcRn inhibitor. In various embodiments, the FcRn inhibitor is selected from the group consisting of efgartigimod, rozanoluximumab, nipocalimab, batoclimab, or orinalolimab. In various embodiments, the therapeutic is an immunosuppressant, complement inhibitor, calcineurin inhibitor, plasmapheresis, IgG protease, proteasome inhibitor and/or inducer of regulatory T cells. In various embodiments, the immunosuppressant is selected from the group consisting of corticosteroids (such as methylprednisolone, prednisolone, or prednisone), rapamycin/sirolimus, or cyclophosphamide, mycophenolate mofetil. In various embodiments, the complement inhibitor is selected from the group consisting of a C1 inhibitor (such as Cinryze, Berinert, Ruconest, sutimlimab (Enjaymo), or Haegarda), C3 inhibitor (such as Pegacetaclplan (Empaveli), Pegacetaclplan injection (Syfovre)), C5 inhibitor (such as eculizumab, ravulizumab, avacopan, pozelimab, nomacopan, zilucoplan, vilobelimab, crovalimab, zimura, cemdisiran, tesidolumab, avdoralimab), Factor D inhibitor (danicopan, vemircopan), or other complement proteins (such as narsoplimab, iptacopan). In various embodiments, the therapeutic is an anti-CD20 CAR T cell therapy, an anti-CD19 CAR T cell therapy, or an anti-BCMA CAR T cell therapy. In various embodiments, the therapeutic is selected from the group consisting of an anti-CD20 antibody (e.g., rituximab), an anti-CD19 antibody, anti-CD40 antibody, CTLA4 Ig (e,g, abatacept). In various embodiments, the calcineurin inhibitor is ciclosporin or tacrolimus. In various embodiments, the proteasome inhibitor is selected from the group consisting of bortezomib, carfilzomib, arsenic trioxide, hydroxychloroquine, or MnTBAP. In some embodiments, the IgG protease is IdeZ, or IdeS (imlifidase). In various embodiments, the Treg inducer is an IL-2 variants/mutein or CDK8/19 inhibitor.

In various embodiments, the one or more therapeutic is administered prior to, concurrently with or subsequent to administration of the carrier particle described herein. In various embodiments, the therapeutic is administered 0.5, 1, 2, 4, 5, 8, 10, 12, 16 or 24 hours prior to administration of the carrier particle including all ranges and values that lie between those ranges. In various embodiments, the therapeutic is administered 1, 2, 3, 4, 5, 6, or 7 days prior to administration of the carrier particle. In various embodiments, the therapeutic is administered 1, 2, 3, or 4 weeks prior to administration of carrier particle. In various embodiments, the therapeutic is administered 1, 2, 3, 4, 5, 6, 7, 8, 9, 10, 11, or 12 months prior to administration of carrier particle. In various embodiments, the therapeutic is administered 1, 2, 3, 4, 5, 6, 7, 8, 9, 10, 11, or 12 years prior to administration of the carrier particle. In various embodiments, the therapeutic is administered concurrently with the carrier particle. In various embodiments, the therapeutic is administered 0.5 1, 2, 4, 5, 8, 10, 12, 16 or 24 hours subsequent to administration of the carrier particle including all ranges and values that lie between those ranges. In various embodiments, the therapeutic is administered 1, 2, 3, 4, 5, 6, or 7 days subsequent to administration of the carrier particle. In various embodiments, the therapeutic is administered 1, 2, 3, or 4 weeks subsequent to administration of carrier particle. In various embodiments, the therapeutic is administered 1, 2, 3, 4, 5, 6, 7, 8, 9, 10, 11, or 12 months subsequent to administration of the carrier particle. In various embodiments, the therapeutic is administered 1, 2, 3, 4, 5, 6, 7, 8, 9, 10, 11, or 12 years subsequent to administration of the carrier particle described herein.

### Screening Methods

It is contemplated that induction of and maintenance of immunological tolerance is monitored in a subject suffering from treated, or about to undergo treatment, with carrier particles as described herein.

Methods of screening for cell types, cytokines or other measures of tolerance from a subject undergoing tolerizing therapy as described herein are known in the art. Methods of assessing tolerance are done using such techniques as flow cytometry, Mass Cytometry (CyTOF), ELISA, ELISPOT, in vitro/ex vivo cell stimulation assays (including, but not limited to, cell proliferation assays, basophil activation test (BAT), macrophage stimulation assays), measuring autoantibodies or measuring Ig serotype, e.g., by ImmunoCap assay.

A list of human metabolites that can be assayed from a biological sample can be found in the literature including in (Psychogios et al., 2011), (Wishart et al., HMDB: the Human Metabolome Database. Nucleic Acids Res. 2007 January; 35(Database issue):D521-6, 2007), and the Human Metabalome Database (HMDB) and are incorporated herein by reference.

One aspect of a subject's immune tolerance status, and immune signature, is determined by analyzing one or more cell-surface proteins from a biological sample(s). In various embodiments, the cell-surface proteins include CD1c, CD2, CD3, CD4, CD5, CD8, CD9, CD10, CD11b, CD11c, CD14, CD15, CD16, CD18, CD19, CD20, CD21, CD22, CD23, CD24, TACI, CD25, CD27, CD28, CD30, CD30L, CD31, CD32, CD32b, CD34, CD33, CD38, CD39, CD40, CD40-L, CD41b, CD42a, CD42b, CD43, CD44, CD45, CD45RA, CD47, CD45RA, CD45RO, CD48, CD52, CD55, CD56, CD58, CD61, CD66b, CD69, CD70, CD72, CD79, CD68, CD84, CD86, CD93, CD94, CD95, CRACC, BLAME, BCMA, CD103, CD107, CD112, CD120a, CD120b, CD123, CD125, CD127, CD134, CD135, CD140a, CD141, CD154, CD155, CD160, CD161, CD163, CD172a, XCR1, CD203c, CD204, CD206, CD207 CD226, CD244, CD267, CD268, CD269, CD355, CD358, CRTH2, NKG2A, NKG2B, NKG2C, NKG2D, NKG2E, NKG2F, NKG2H, KIR2DL1, KIR2DL2, KIR2DL3, KIR2DL5A, KIR2DL5B, KIR3DL1, KIR3DL2, KIR3DL3, KIR3DL4, KIR2DS1, KIR2DS2, KIR2DS3, KIR2DS4, KIR2DS5, DAP12, KIR3DS, NKp44, NKp46, TCR, BCR, Integrins, FcβεRI, MHC-I, MHC-II, IL-1R, IL-2Rα, IL-2Rβ, IL-2Rγ, IL-3Rα, CSF2RB, IL-4R, IL-5Rα, CSF2RB, IL-6Rα, gp130, IL-7Rα, IL-9R, IL-10R, IL-12Rβ1, IL-12Rβ2, IL-13Rα1, IL-13Rα2, IL-15Rα, IL-21R, IL-23R, IL-27Rα, IL-31Rα, OSMR, CSF-1R, cell-surface IL-15, IL-10Rα, IL-10Rβ, IL-20Rα, IL-20Rβ, IL-22Rα1, IL-22Rα2, IL-22Rβ, IL-28RA, PD-1, PD-1H, BTLA, CTLA-4, PD-L1, PD-L2, 2B4, B7-1, B7-2, B7-H1, B7-H4, B7-DC, DR3, LIGHT, LAIR, LTα1β2, LTβR, TIM-1, TIM-3, TIM-4, TIGIT, LAG-3, ICOS, ICOS-L, SLAM, SLAMF2, OX-40, OX-40L, GITR, GITRL, TL1A, HVEM, 41-BB, 41BB-L, TL-1A, TRAF1, TRAF2, TRAF3, TRAF5, BAFF, BAFF-R, APRIL, TRAIL, RANK, AITR, TRAMP, CCR1, CCR2, CCR3, CCR4, CCR5, CCR6, CCR7, CCR8, CCR9, CCR10, CCR11, CXCR1, CXCR2, CXCR3, CXCR4, CXCR5, CXCR6, CXCR7, CLECL9a, DC-SIGN, IGSF4A, SIGLEC, EGFR, PDGFR, VEGFR, FAP, α-SMA, FAS, FAS-L, FC, ICAM-1, ICAM-2, ICAM-3, ICAM-4, ICAM-5, PECAM-1, MICA, MICB, UL16, ULBP1, ULBP2, ILBP3, ULBP4, ULBP5, ULBP6, MULT1, RAE1 α,β,γ,δ, and ε, H60a, H60b, H60c, GPR15, ST2, and/or combinations thereof. Integrins include α1, α2, αIIb, α3, α4, α5, α6, α7, α8, α9, α10, α11, αD, αE, αL, αM, αV, αX, β1, β2, β3, β4, β5, β6, β7, β8 and/or combinations thereof. TCR include α, β, γ, δ, ε, ζ chains and/or combinations thereof. Several methods have been described in the literature for assaying of cell-surface protein expression, including Flow Cytometry and Mass Cytometry (CyTOF).

In certain embodiments, the subject's tolerance status is determined by analyzing nucleic acids from the biological sample(s). In various embodiments, the nucleic acids are DNA and/or RNA, including, but not limited to, single stranded DNA, double stranded DNA, mRNA, rRNA, tRNA, siRNA, miRNA, long non-coding RNAs (long ncRNAs, IncRNA), and non-coding RNA (ncRNA), mitochondrial RNA. In various embodiments, the subject's immune tolerance status is determined by assaying gene expression from the biological sample(s). In various embodiments, the immune tolerance status is determined by assaying gene expression associated with immune function, an antibody, foreign body response, metabolism, apoptosis, cell death, necrosis, ferroptosis, autophagy, cell migration, endocytosis, phagocytosis, pinocytosis, tight-junction regulation, cell adhesion, differentiation, and/or combinations thereof. In various embodiments, the immune tolerance status is determined by assaying gene expression associated with immune suppression. In various embodiments, the immune tolerance status is determined by assaying gene expression associated with immune activation. In various embodiments, the immune tolerance status is determined by assaying gene expression associated with immune regulatory functions. In various embodiments, nucleic acid analysis is used to generate an immune tolerance signature. Several methodologies have been described in the literature for high-throughput gene expression analysis including RNA sequencing (RNA-seq), single-cell RNA sequencing (scRNA-seq), exome sequencing, and microarray-based analyses.

The biological sample is optionally assayed after in vivo and/or ex vivo stimulation with one or more stimuli such as an antigen, an allergen, and one or more activating agents. It is contemplated that the T cells, B cells, and immunoglobulins used in the assay are antigen specific. Exemplary T cells include effector memory T cells, antigen specific T cells, activated antigen specific T cells, Th1 cells, pathogenic Th2a+ cells, Th17 cells, T follicular helper (TFH) cells, TH0 cells, or other antigen-specific T cells. B cells include effector B cells, memory B cells, plasma cells, and regulatory B (Breg) cells.

In various embodiments, the immune tolerance status of the subject is determined by obtaining one or more samples, e.g., whole blood, from the subject pre-dose on the day of the first TIMP administration (Day 1), and at a date(s) after administration. Whole blood can then be processed to isolate peripheral blood mononuclear cells (PBMCs), basophils, neutrophils, plasma, and serum for downstream analyses. Assay of cells isolated from one or more samples collected from the subject and analyzed.

### Pharmaceutical Formulations

Pharmaceutical compositions of the present disclosure containing the TIMP described herein and an antigen may contain pharmaceutically acceptable carriers or additives depending on the route of administration. Examples of such carriers or additives include water, a pharmaceutically acceptable organic solvent, collagen, polyvinyl alcohol, polyvinylpyrrolidone, a carbox-yvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline®, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present disclosure.

Formulation of the pharmaceutical composition will vary according to the route of administration selected (e.g., solution, emulsion). An appropriate composition comprising the therapeutic to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils. Intravenous vehicles can include various additives, preservatives, or fluid, nutrient or electrolyte replenishers.

A variety of aqueous carriers, e.g., sterile phosphate buffered saline solutions, bacteriostatic water, water, buffered water, 0.4% saline, 0.3% glycine, and the like, and may include other proteins for enhanced stability, such as albumin, lipoprotein, globulin, etc., subjected to mild chemical modifications or the like.

Therapeutic formulations of the inhibitors are prepared for storage by mixing the inhibitor having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl para-bens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations to be used for in vivo administration must be sterile. This is readily accomplished by filtration through sterile filtration membranes.

Aqueous suspensions may contain the active compound in admixture with excipients suitable for the manufacture of aqueous suspensions. Such excipients are suspending agents, for example sodium carboxymethylcellulose, methylcellulose, hydroxypropylmethylcellulose, sodium alginate, polyvinylpyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyl-eneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate.

The TIMP comprising antigen as described herein can be lyophilized for storage and reconstituted in a suitable carrier prior to use.

Solid dosage forms for oral administration include capsules, tablets, pills, powders, and granules. In such solid dosage forms, the modified particles are mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

### Kits

The disclosure also provides kits which comprise one or more compounds or compositions packaged in a manner which facilitates their use to practice methods of the disclosure. In one embodiment, such a kit includes a compound or composition described herein (e.g., a composition comprising a TIMP alone or in combination with another agent), packaged in a container such as a sealed bottle or vessel, with a label affixed to the container or included in the package that describes use of the compound or composition in practicing the method. Preferably, the compound or composition is packaged in a unit dosage form. The kit may further include a device suitable for administering the composition according to a specific route of administration or for practicing a screening assay. Preferably, the kit contains a label that describes use of the particle compositions.

Additional aspects and details of the disclosure will be apparent from the following examples, which are intended to be illustrative rather than limiting.

### EXAMPLES

### Example 1—Efficacy of TIMPs at Improving Expression After AAV8-GFP Administration and Enabling Re-Dosing of AAV8-GFP

The efficacy of TIMPs encapsulating recombinant eGFP (CNP-GFP) and AAV8 VP1 capsid protein (CNP-VP1)(CNP-GFP+CNP-VP1) at inducing antigen specific tolerance and improving transgene expression upon initial administration and upon re-dosing with rAAV8-GFP vector was examined.

The particles used in this study had an average diameter of 400-800 nm and a zeta potential of between −30 and −80 mV.

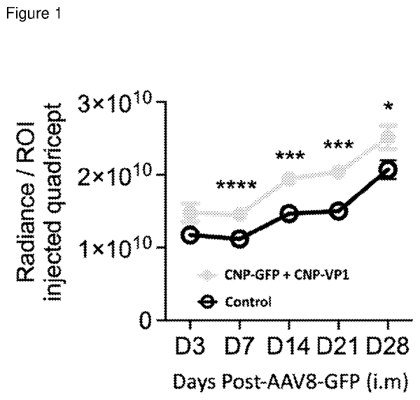

Female BALB/c mice (6-8 weeks of age, n=10 per group) were injected intravenously with CNP-GFP (1.25 mg/mouse) and CNP-VP1 (1.25 mg/mouse) or Unloaded CNPs (Control) on Day −7 and Day 0. Mice were administered rAAV8-eGFP via intramuscular injection in the left quadricep on Day 0. Mice were monitored for transgene eGFP expression in the primed left quadricep on Day 0, 3, 7, 14, 21, and 28 by transcutaneous fluorescence spectroscopy. As shown in FIG. 1, treatment with CNP-GFP+CNP-VP1 led to significantly higher GFP expression when compared to Control (*p<0.05; ***p<0.001; ****p<0.0001).

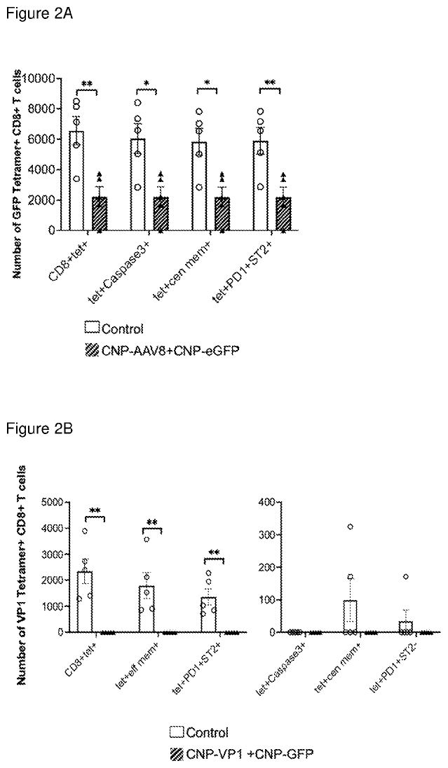

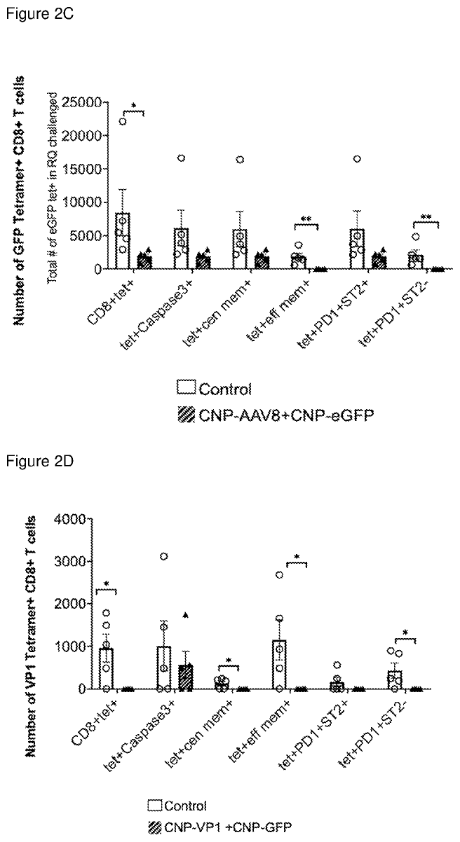

On Day 28, mice were re-dosed with AAV8-GFP intramuscularly on the contralateral right quadricep (N=5 from each group). Seven days after re-dosing (Day 35), mice were sacrificed and both the right and left quadriceps were harvested for analysis. Frequencies and total cell counts of AAV8-VP1 and GFP specific CD8⁺ T cells were evaluated by flow cytometry using tetramer staining. As shown in FIG. 2, treatment with CNP-GFP+CNP-VP1 led to a significant reduction in the number of VP1 and GFP specific CD8+ T cells in both the initial tissue of AAV8-GFP administration (left quadricep) and at the tissue of AAV8-GFP re-dosing (right quadricep) (*p<0.05; **p<0.01).

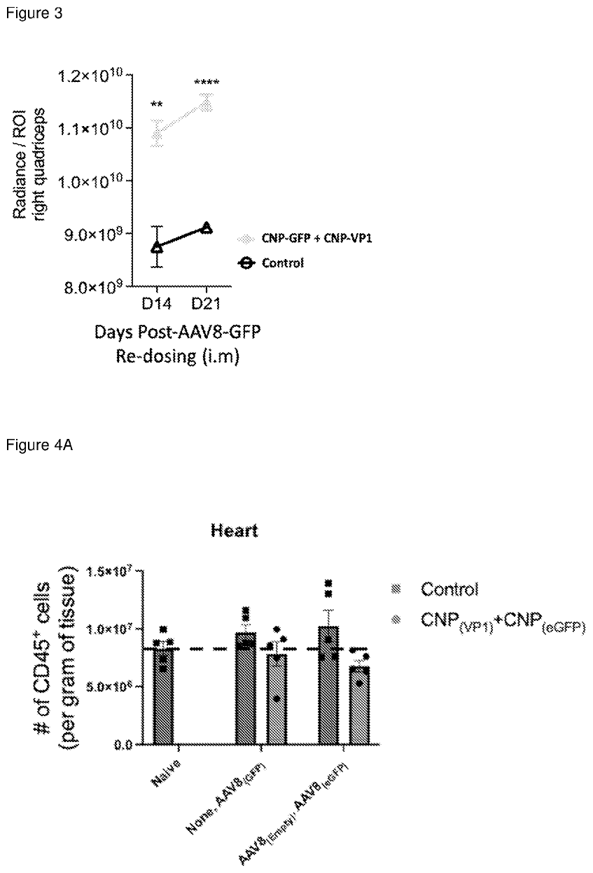

On Day 35, the remaining mice in each treatment group (N=5 per group) were re-dosed with AAV8-GFP intramuscularly on the contralateral right quadricep and monitored for eGFP expression in the right quadricep by transcutaneous fluorescence spectroscopy. As shown in FIG. 3, mice treated with CNP-GFP+CNP-VP1 demonstrated significantly higher eGFP expression in the right quadricep on Days 14 and 21 after AAV8-GFP re-dosing when compared to Control (**p<0.01; ****p<0.0001). Relative to Control treatment, CNP-GFP+CNP-VP1 treatment led to a 58% increase in GFP expression from Day 14 to Day 21.

Based on these results, treatment with CNP-GFP+CNP-VP1 may inhibit the production of anti-VP1 and anti-GFP antibodies. The inhibition of the antibody response is measured from blood using ELISA.

Results of this study demonstrate that TIMPs encapsulating AAV8 VP1 capsid protein and the transgene product are effective at inducing antigen specific tolerance leading to improved transgene expression and enable re-dosing of AAV8-GFP.

### Example 2—Intravenous Administration of TIMPs Encapsulating AAV8-GFP Leads to Systemic Tolerance

The efficacy of TIMPs encapsulating recombinant eGFP (CNP-GFP) and AAV8 VP1 capsid protein (CNP-VP1) at inducing antigen specific tolerance upon intravenous administration in a therapeutic mouse model was examined.

The particles used in this study had an average diameter of 400-800 nm and a zeta potential between −30 and −80 mV.

C57BL/6 mice (age 6-8 weeks, n=5) were primed with either PBS or empty AAV8 capsid on day 0. Mice were administered 2 intravenous doses of CNP-GFP (1.25 mg/mouse)+CNP-VP1 (1.25 mg/mouse) on day 14 and day 21. Mice were challenged by intravenous AAV8-GFP administration on day 28. Mice were sacrificed and the final readout was taken on day 53.

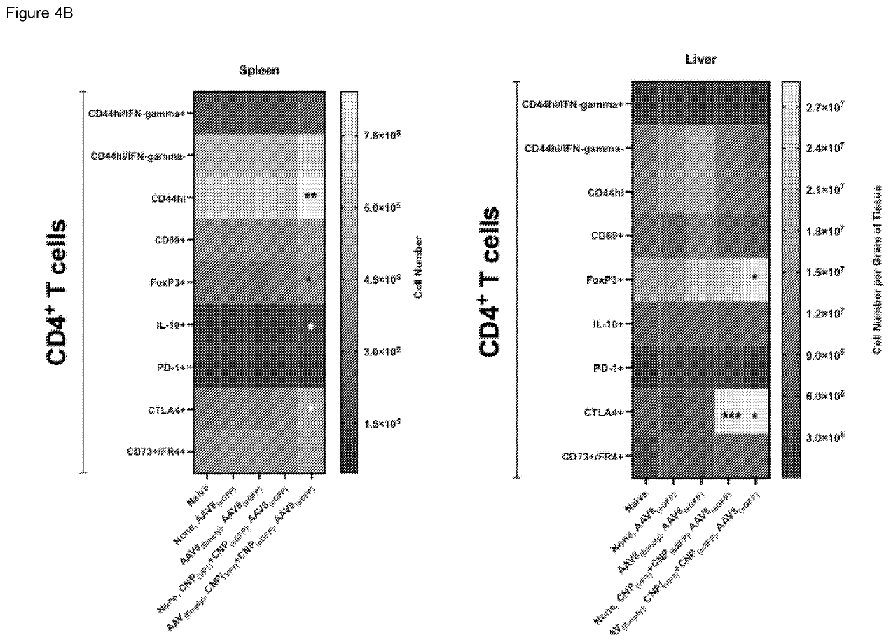

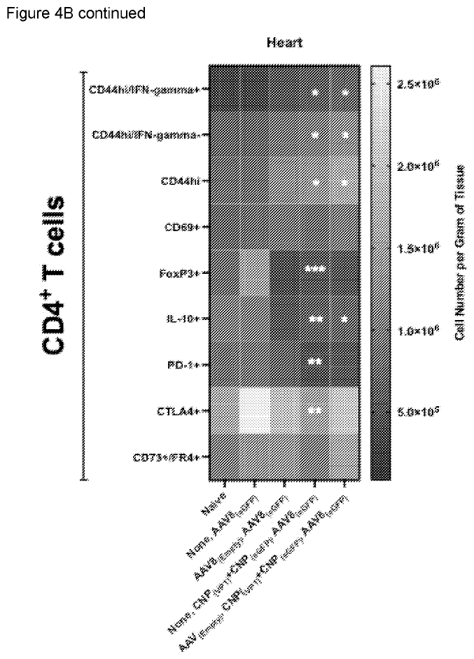

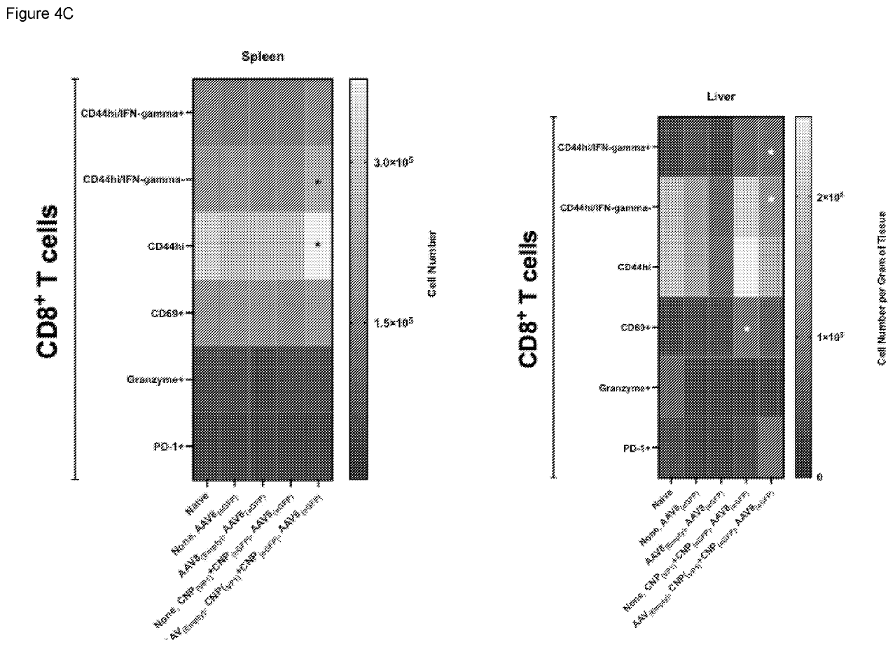

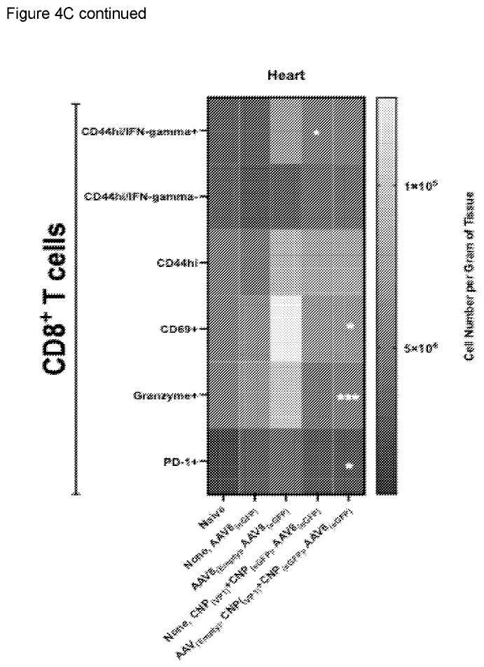

Mice treated with CNPs have decreased immune infiltrate in the heart compared to control mice (FIG. 4A).

As shown in FIG. 4B, CD4+ T cells obtained from mice treated with CNPs show increased regulatory phenotypes in the spleen, liver, and heart (FIG. 4B). Mice treated with CNPs have an increased number of CD4+ T-cells expressing regulatory markers (Foxp3+ and Ctla4+) in the spleen and liver as compared with control treated mice. Mice receiving CNP therapy also had increased numbers of CD4+ T cells expressing regulatory cytokine IL-10+ and CD44hi memory marker in the spleen (FIG. 4B). Mice treated with CNPs have increased CD4+ T cells with memory phenotypes (CD44hi, CD44hi IFN-gamma+, and CD44hi IFN-gamma−) and increased regulatory cells (CD4+Foxp3+, CD4+PD-1+, CD4+CTLA4+, CD4+IL-10+) in the heart. (*p<0.05; **p<0.01).

As shown in FIG. 4C, mice that were therapeutically treated with CNP and re-dosed with AAV have decreased numbers of effector CD8+ T cells (CD8+granzyme+, CD8+PD-1+, CD8+CD69+) infiltrating the heart when compared to control-treated mice (FIG. 4C). (*p<0.05; **p<0.01; ***p<0.001).

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize or be able to ascertain many equivalents to the specific embodiments of the invention described herein. Such equivalents are intended to be encompassed by the following claims.

### REFERENCES

1. Ashtari M, Cyckowski LL, Monroe JF, et al. The human visual cortex responds to gene therapy-mediated recovery of retinal function. J Clin Invest. 2011;121 (6):2160-2168.
2. Bevan AK, Duque S, Foust KD, et al. Systemic gene delivery in large species for targeting spinal cord, brain, and peripheral tissues for pediatric disorders. Mol Ther. 2011;19(11):1971-1980.
3. Evans CH, Ghivizzani SC, Robbins PD. Gene Delivery to Joints by Intra-Articular Injection. Hum Gene Ther. 2018;29(1):2-14.
4. Freedman JD, Hagel J, Scott EM, et al. Oncolytic adenovirus expressing bispecific antibody targets T-cell cytotoxicity in cancer biopsies. EMBO Mol Med. 2017;9(8):1067-1087.
5. Nienhuis AW, Nathwani AC, Davidoff AM. Gene Therapy for Hemophilia. Mol Ther. 2017;25(5):1163-1167.
6. Piguet F, Alves S, Cartier N. Clinical Gene Therapy for Neurodegenerative Diseases: Past, Present, and Future. Hum Gene Ther. 2017;28(11):988-1003.
7. Venkatesha SH, Dudics S, Acharya B, Moudgil KD. Cytokine-modulating strategies and newer cytokine targets for arthritis therapy. Int J Mol Sci. 2014;16(1):887-906.
8. Lundstrom K. New frontiers in oncolytic viruses: optimizing and selecting for virus strains with improved efficacy. Biologics. 2018;12:43-60.
9. Conry RM, Westbrook B, McKee S, Norwood TG. Talimogene laherparepvec: First in class oncolytic virotherapy. Hum Vaccin Immunother. 2018;14(4):839-846.
10. Lundstrom K. Viral Vectors in Gene Therapy. Diseases. 2018;6(2).
11. Guenther CM, Kuypers BE, Lam MT, Robinson TM, Zhao J, Suh J. Synthetic virology: engineering viruses for gene delivery. Wiley Interdiscip Rev Nanomed Nanobiotechnol. 2014;6(6):548-558.
12. Mingozzi F, Maus MV, Hui DJ, et al. CD8(+) T-cell responses to adeno-associated virus capsid in humans. Nat Med. 2007;13(4):419-422.
13. Mingozzi F, High KA. Immune responses to AAV vectors: overcoming barriers to successful gene therapy. Blood. 2013;122(1):23-36.
14. Colella P, Ronzitti G, Mingozzi F. Emerging Issues in AAV-Mediated In Vivo Gene Therapy. Mol Ther Methods Clin Dev. 2018;8:87-104.
15. Arruda VR, Favaro P, Finn JD. Strategies to modulate immune responses: a new frontier for gene therapy. Mol Ther. 2009;17(9):1492-1503.
16. Meliani A, Boisgerault F, Hardet R, et al. Antigen-selective modulation of AAV immunogenicity with tolerogenic rapamycin nanoparticles enables successful vector re-administration. Nat Commun. 2018;9(1):4098.
17. Sánchez A, Mejía SP, Orozco J. Recent Advances in Polymeric Nanoparticle-Encapsulated Drugs against Intracellular Infections. Molecules. 2020;25(16).
18. Zielińska A, Carreiró F, Oliveira AM, et al. Polymeric Nanoparticles: Production, Characterization, Toxicology and Ecotoxicology. Molecules. 2020;25(16).
19. Majowicz A, Salas D, Zabaleta N, et al. Successful Repeated Hepatic Gene Delivery in Mice and Non-human Primates Achieved by Sequential Administration of AAV5^{ch} and AAV1. Mol Ther. 2017;25(8):1831-1842.
20. Greig JA, Calcedo R, Grant RL, et al. Intramuscular administration of AAV overcomes pre-existing neutralizing antibodies in rhesus macaques. Vaccine. 2016;34(50):6323-6329.
21. Stubbs AC, Martin KS, Coeshott C, et al. Whole recombinant yeast vaccine activates dendritic cells and elicits protective cell-mediated immunity. Nat Med. 2001;7(5):625-629.
22. Munagala R, Aqil F, Jeyabalan J, et al. Exosome-mediated delivery of RNA and DNA for gene therapy. Cancer Lett. 2021;505:58-72
23. Kim SM, Yang Y, Oh SJ, Hong Y, Seo M, Jang M. Cancer-derived exosomes as a delivery platform of CRISPR/Cas9 confer cancer cell tropism-dependent targeting. J Control Release. 2017;266:8-16.
24. Byun HM, Suh D, Yoon H, et al. Erythrocyte ghost-mediated gene delivery for prolonged and blood-targeted expression. Gene Ther. 2004;11(5):492-496
25. Ihler GM, Glew RH, Schnure FW. Enzyme loading of erythrocytes. Proc Natl Acad Sci USA. 1973;70(9):2663-2666.

We claim:

1. A composition comprising negatively charged particles encapsulating an antigen, wherein the antigen is one or more gene therapy vector antigens, portions thereof, or combinations thereof and/or one or more transgene protein products produced by a gene therapy vector(s), portions thereof, or combinations thereof.

2. The composition of claim 1, wherein the particle comprises poly (lactide-co-glycolide) (PLG), polylactic acid (PLA), a co-polymer of PLG and PLA (PLGA), polycaprolactone (PCL), polystyrene, polyethylene glycol (PEG), chitosan, a polysaccharide, one or more lipids, iron, zinc, cadmium, gold or silver.

3. The composition of claim 1 or 2, wherein the particle has a zeta potential between 0 mV and −100 mV.

4. The composition of claim 3, wherein the particle has a zeta potential between −30 and −80 mV.

5. The composition of any one of claims 1-4, wherein the particle size is between 100 nm and 1000 nm.

6. The composition of claim 5, wherein the particle size is between 400-800 nm

7. The composition of any one of claims 1-5, wherein the antigen comprises one or more proteins, peptides or antigenic epitopes thereof.

8. The composition of any one of claims 1-7, wherein the gene therapy vector is a viral vector.

9. The composition of 8, wherein the viral vector is an adenovirus, adeno-associated virus (AAV), herpes simplex virus, hepatitis B virus, lentivirus, retrovirus, alphavirus, flavivirus, rhabdovirus, measles virus, Newcastle disease virus, coxsackieviruses, transfusion transmitted viruses, anellosomes, human papilloma viruses, poxvirus, vaccinia virus, modified Ankara virus, vesicular stomatitis virus, picornavirus, tobacco mosaic virus, cowpea mosaic virus, cowpea chlorotic mottle virus, physalis mosaic virus, red clover necrotic mosaic virus, potato virus x, comovirus, chicken anemia virus or cucumber mosaic virus.

10. The composition of claim 9, wherein the AAV is AAV-1, AAV-2, AAV-3, AAV-4, AAV-5, AAV-6, AAV-7, AAV-8, AAV-9, AAV-10, AAV-12, Anc80, a synthetic AAV, combinations, engineered, or modified versions thereof.

11. The composition of any one of claims 1-10, wherein the antigen is associated with one or more viral vector capsid proteins, peptides, and/or antigenic epitopes thereof.

12. The composition of claim 11, wherein the capsid protein is VP-1, VP-2, VP-3, combinations, or portions thereof.

13. The composition of any one of claims 1-12, wherein the transgene protein product is a cytokine, a chemokine, a hormone, a growth factor, an enzyme, or an antibody.

14. The composition of claim 13, wherein the antibody is a monoclonal antibody.

15. A method of inducing tolerance to a subject in need thereof comprising administering to a subject a composition comprising negatively charged particles encapsulating an antigen, wherein the antigen is one or more gene therapy vector antigens and/or one or more transgene protein products produced by the gene therapy vector(s), portions, or combinations thereof.

16. The method of claim 15, wherein the negatively charged particles encapsulating one or more gene therapy vector antigens are administered in combination with negatively charged particles encapsulating one or more transgene protein products produced by the gene therapy vector(s).

17. The method of claim 16, wherein the negatively charged particles encapsulating one or more gene therapy vector antigens are administered before, concomitantly or after the administration of negatively charged particles encapsulating one or more transgene protein products produced by the gene therapy vector(s).

18. The method of any one of claims 15-17, wherein the particle comprises poly (lactide-co-glycolide) (PLG), polylactic acid (PLA), a co-polymer of PLG and PLA (PLGA), polycaprolactone (PCL), polyethylene glycol (PEG), polystyrene, chitosan, a polysaccharide or one or more lipids, iron, zinc, cadmium, gold or silver.

19. The method of any one of claims 15-18, wherein particles have a negative zeta potential.

20. The method of any one of claims 15-19, wherein the particle has a zeta potential between 0 mV and −100 mV.

21. The method of any one of claims 15-20, wherein the particle has a zeta potential between −30 mV and −100 mV.

22. The method of any one of claims 15-21, wherein the size of the particle is between 100 nm and 1000 nm.

23. The method of any one of claims 15-22, wherein the size of the particle is between 400 nm and 800 nm.

24. The method of claim of any one of claims 15-23, wherein the antigen comprises a protein, peptide or one or more antigenic epitopes thereof.

25. The method of any one of claims 15-24, wherein the gene therapy vector is a viral vector.

26. The method of claim 25, wherein the viral vector is an adenovirus, adeno-associated virus (AAV), herpes simplex virus, hepatitis B virus, lentivirus, retrovirus, alphavirus, flavivirus, rhabdovirus, measles virus, Newcastle disease virus, coxsackieviruses, transfusion transmitted viruses, anellosomes, human papilloma viruses, poxvirus, vaccinia virus, modified Ankara virus, vesicular stomatitis virus, picornavirus, tobacco mosaic virus, cowpea mosaic virus, cowpea chlorotic mottle virus, physalis mosaic virus, red clover necrotic mosaic virus, potato virus x, comovirus, chicken anemia virus or cucumber mosaic virus.

27. The method of claim 26, wherein the AAV is selected from the group consisting of AAV-1, AAV-2, AAV-3, AAV-4, AAV-5, AAV-6, AAV-7, AAV-8, AAV-9, AAV-10, AAV-12, Anc80, a synthetic AAV, combinations or engineered versions thereof.

28. The method of any one of claims 15-27, wherein the antigen is associated with one or more viral vector capsid proteins, peptides, and/or antigenic epitopes thereof.

29. The method of claim 28, wherein the capsid protein is VP-1, VP-2, VP-3, combinations or portions thereof.

30. The method of any one of claims 15-29, wherein the subject has cancer, autoimmune disease, allergy, cardiovascular disease, metabolic disease, diabetes, enzyme deficiency, protein deficiency, cystic fibrosis, hematologic disorder, beta-thalassemia, sickle-cell disease, Hemophilia A, Hemophilia B, lysosomal storage disease, Fabry disease, Gaucher disease, Pompe disease, Niemann-Pick disease, Tay-Sachs disease, macular degeneration, mucopolysaccharidosis, venous thrombosis, von Willebrand disease, purpura fulminans, growth-hormone deficiency, gangliosidosis, alkaline hypophosphatasia, cholesterol ester storage disease, hyperuricemia, Duchenne Muscular Dystrophy, Huntington's disease, Parkinson's, Alzheimer's disease, choroiderimia, Stargardt Disease, Batten disease, spinocerebellar ataxia, ALS, frontotemporal lobar degeneration, ornithine transcarbamylase deficiency, retinitis pigmentosa, RPE-65 mutation-associated diseases, epidermolysis bullosa, recessive dystrophic epidermolysis bullosa, spinal muscular atrophy, phenylketonuria (PKU), X-linked myotubular myopathy, Crigler-Najjar syndrome, Catecholaminergic polymorphic ventricular tachycardia, glycogen storage disease type 1, alpha-mannosidosis, Fragile X-syndrome, arginase deficiency, X-linked chronic granulomatous disease, adenosine deaminase deficiency, Leber's congenital amaurosis, lipoprotein lipase deficiency, cerebral adrenoleukodystrophy, metachromatic leukodystrophy, Fanconi anemia, achromatopsia, scleroderma, Osteogenesis imperfecta, coronary artery disease, tyrosinemia, peripheral neuropathy, optic neuropathy, coronary artey disease, Respiratory syncytial virus (RSV)-mediated lower respiratory tract disease, Danon disease, severe leukocyte adhesion deficiency, pyruvate kinase deficiency, Charcot Marie Tooth disease, Wiskott Aldrich syndrome, Alpha-synuclein tauopathies, refractory angina due to myocardial ischemia, myotonic dystrophy type I, claudication, peripheral artery disease, methylmalonic acidemia, Sucrase-isomaltase deficiency, Niemann-Pick type B disease, α1-PI deficiency, hereditary angioedema, Fibrinogen deficiency, Factor VIIa deficiency, Factor X deficiency, Factor XI deficiency, Factor XII deficiency, Protein C deficiency, Anti-thrombin III deficiency, MPS I, MPS II, MPS III, MPS IV, MPS VI, MPS VII, MPS IX, calcification/ossification disorders, ENPP1 deficiency, ENPP3 deficiency, ABCC6 deficiency, aromatic L-amino acid decarboxylase deficiency, Angelman syndrome, hyperphenylalaninaemia, dementia, Rett syndrome and Usher syndrome.

31. The method of claim 30, wherein the subject has a cancer selected from the group consisting of brain cancer, skin cancer, eye cancer, breast cancer, prostate cancer, lung cancer, esophageal cancer, head and neck cancer, cervical cancer, liver cancer, colon cancer, bone cancer, uterine cancer, ovarian cancer, bladder cancer, stomach cancer, oral cancer, thyroid cancer, kidney cancer, testicular cancer, leukemia, lymphoma, melanoma and mesothelioma.

32. The method of claim 30, wherein the subject has an autoimmune disease selected from the group consisting of multiple sclerosis, Addison's disease, ankylosing spondylitis, alopecia, osteoarthritis, psoriatic arthritis, scleroderma, type-I diabetes, rheumatoid arthritis, thyroiditis, systemic lupus erythematosus, Reynaud's syndrome, Behcets syndrome, Sjorgen's syndrome, autoimmune uveitis, Eaton Lamberts disease, autoimmune myocarditis, inflammatory bowel disease, amyotrophic lateral Iclerosis (ALS), systemic lupus erythematosus, neuromyelitis optica, idiopathic thrombocytopenia purpura, thrombotic thrombocytopenia purpura, membranous nephropathy, bullous phemphigoid, phemphigus vulgaris, myasthenia gravis, Celiac disease, ulcerative colitis, Crohn's disease, erythema nodosa, glomerulonephritis, Goodpasture's syndrome, granulomatosis, Grave's disease, Guillain-Barre syndrome, Hashimoto disease, hemolytic anemia, Kawasaki Disease, mixed connective tissue disease, multifocal motor neuropathy, peripheral biliary cirrhosis, polyangiitis overlap syndrome, scleroderma type 1, sclerosis cholangitis, Siffman syndrome, Takayasu arteritis, vitiligo or Wegeners granulomatosis.

33. The method of claim 30, wherein the subject has an allergy selected from the group consisting of peanut allergy, tree nut allergy, milk allergy, egg allergy, fish allergy, wheat allergy, celery allergy or peach allergy. In various embodiments the environmental allergy is pollen allergy, dust allergy, pet dander allergy or mold allergy.

34. The method of any one of claims 15-33, wherein the composition is administered before, after or concomitantly with the gene therapy vector.

35. The method of any one of claims 15-34, wherein administration of the composition reduces an immune response against the gene therapy vector and/or the transgene protein product produced by the vector.

36. The method of claim 35, wherein the immune response is an inflammatory immune response.

37. The method of any one of claims 35-36, wherein the immune response is a humoral immune response.

38. The method of any one of claims 35-36, wherein the immune response is an adaptive immune response.

39. The method of any one of claims 35-36, wherein the immune response is an innate immune response.

40. The method of any one of claims 35-39, wherein the immune response is a T cell, B cell, monocyte, macrophage, neutrophil, basophil, or eosinophil response.

41. The method of any one of claims 35-40, wherein the immune response is an antibody response.

42. The method of claim 41, where the antibody response is the formation of neutralizing antibodies against the gene therapy vector and/or the transgene protein produced by the vector.

43. The method of any one of claims 15-42, wherein administration of the composition induces an immune regulator response against the gene therapy vector and/or the transgene protein product produced by the vector.

44. The method of claim 43, wherein the immune regulator response is regulatory T cells, B cells, monocytes, and/or macrophages.

45. The method of claim 43, wherein the immune regulator response is antigen specific Treg, Tr1, Mreg, and/or Breg cells.

46. The method of any one of claims 15-45, wherein the composition is administered intravenously, intramuscularly, ocularly, intraperitoneally, transdermally, nasally, orally and/or subcutaneously.

47. A composition comprising liposomes encapsulating one or more gene therapy vector antigens, portions, or combinations thereof.

48. A composition comprising liposomes encapsulating therapeutic proteins produced by the gene therapy vector and/or a portion thereof.

49. The composition of any one of claims 47-48, wherein the liposome has a negative zeta potential.

50. The composition of claim 49, wherein the negative zeta potential is between −100 mV to 0 mV.

51. The composition of claim 50, wherein the negative zeta potential is between −80 mV to −30 mV.

52. The composition of any one of claims 47-51, wherein the gene therapy vector antigen is selected from the group consisting of adenovirus, adeno-associated virus (AAV), AAV-1, AAV-2, AAV-3, AAV-4, AAV-5, AAV-6, AAV-7, AAV-8, AAV-9, AAV-10, AAV-12, Anc80, synthetic AAV, combinations or engineered versions thereof, AAV capsid protein VP1, AAV capsid protein VP2, AAV capsid protein VP3, herpes simplex virus, hepatitis B virus, lentivirus, retrovirus, alphavirus, flavivirus, rhabdovirus, measles virus, Newcastle disease virus, coxsackieviruse, transfusion transmitted viruses, anellovirus, human papilloma virus, poxvirus, vaccinia virus, modified Ankara virus, vesicular stomatitis virus, picornavirus, tobacco mosaic virus, cowpea mosaic virus, cowpea chlorotic mottle virus, physalis mosaic virus, Red clover necrotic mosaic virus, potato virus x, comovirus, chicken anemia virus, cucumber mosaic virus, oncolytic virus, chimeric virus, a synthetic virus, a mosaic virus, a pseudotyped virus, bacteria, bacteriophage, yeast, exosome, or erythrocyte

53. The composition of any one of claims 47-52, wherein the therapeutic protein produced by the gene therapy vector and/or a portion thereof is selected from the group consisting of a cytokine, a chemokine, a hormone, a growth factor, an enzyme, or an antibody.

54. The composition of any one of claims 47-53, wherein the liposome is between 100-1000 nm.

55. A method of inducing tolerance in a subject in need thereof comprising administering to the subject a composition of any one of claims 47-54.

56. A composition according to any one of claim 1-14 or 47-54 comprising a negatively charged particle encapsulating an antigen, wherein the antigen is one or more gene therapy vector antigens and/or one or more transgene protein products produced by the gene therapy vector(s), portions, or combinations thereof for use in inducing tolerance in a subject.

57. Use of a composition according to any one of claim 1-14 or 47-54 comprising a negatively charged particle encapsulating an antigen, wherein the antigen is one or more gene therapy vector antigens and/or one or more transgene protein products produced by the gene therapy vector(s), portions, or combinations thereof for use in the preparation of a medicament for inducing tolerance in a subject.

58. A method of inducing tolerance to a subject in need thereof comprising administering to a subject a composition according to any one of claim 1-14 or 47-54 comprising a negatively charged particle encapsulating an antigen, wherein the antigen is one or more gene therapy vector antigens and/or one or more transgene protein products produced by the gene therapy vector(s), portions, or combinations thereof in combination with one or more therapeutic.

59. The method of claim 58, wherein the therapeutic blocks or inhibits innate immune responses, complement responses, B cell responses, and/or T cell responses against the gene therapy vector and/or transgene protein produced by the gene therapy vector.

60. The method of claim 58 or 59, wherein the therapeutic is selected from the group consisting of immunosuppressant, complement inhibitor, calcineurin inhibitor, plasmapheresis, IgG protease, proteasome inhibitor and/or inducer of regulatory T cells.

61. The method of any one of claims 58-60, wherein the therapeutic is an FcRn inhibitor.

62. The method of any one of claims 58-60, wherein the therapeutic is a corticosteroid, rapamycin/sirolimus, cyclophosphamide, or mycophenolate mofetil.

63. The method of any one of claims 58-60, wherein the therapeutic is an anti-CD20 CAR T cell therapy, an anti-CD19 CAR T cell therapy, or an anti-BCMA CAR T cell therapy.

64. The method of any one of claims 58-60, wherein the therapeutic is an anti-CD20 antibody, an anti-CD19 antibody, anti-CD40 antibody, or CTLA4 Ig.

65. The method of any one of claims 58-64, wherein the one or more therapeutic is administered prior to, concurrently with, or subsequent to administration of the composition according to any one of claim 1-14 or 47-54 comprising a negatively charged particle encapsulating an antigen, wherein the antigen is one or more gene therapy vector antigens and/or one or more transgene protein products produced by the gene therapy vector(s), portions, or combinations thereof.
